(19) United States
(12) Patent Application Publication
(10) Pub. No.: US 2005/0256077 A1
(43) Pub. Date: Nov. 17, 2005

(54) Gastrointestinal stem cells and uses thereof

(76) Inventor: Susan J. Henning, Houston, TX (US)

Correspondence Address: Fulbright & Jaworski L.L.P., Suite 5100, 1301 McKinney, Houston, TX, 77010-3095, US

(21) Appl. No.: 11/094,456

(22) Filed: Mar. 30, 2005

Related U.S. Application Data
(60) Provisional application No. 60/557,797, filed on Mar. 30, 2004.

(51) Int. Cl. A61K 48/00

(52) U.S. Cl. 514/44

(57) ABSTRACT

The present invention relates to compositions and methods concerning isolated gastrointestinal stem cells. Particularly, the invention provides isolated gastrointestinal stem cells comprising a CD45 negative marker, a collagen IV negative marker, and that is Msi-1 positive. In particular embodiments the isolated cells are comprised in a single cell suspension. In other particular embodiments, the isolated cells are utilized for therapeutic purposes, such as for a gene therapy vector and/or for replenishing stem cells in a gastrointestinal tract in need thereof.

The present invention claims priority to U.S. Provisional Patent Application Ser. No. 60/557,797, filed Mar. 30, 2004, which is incorporated by reference herein in its entirety.

### STATEMENT REGARDING FEDERALLY SPONSORED RESEARCH OR DEVELOPMENT

The present invention was developed at least in part from funding provided by the National Institutes of Health Grant No. R21 DK61132, by the National Institutes of Health Grant No. T32DK07664, and by the National Institutes of Health Grant No. P30DK56338. The United States Government may have certain rights in the invention.

### FIELD OF THE INVENTION

The present invention regards the fields of cell biology, molecular biology, and medicine. In particular, the invention is related to the field of intestinal stem cells and therapeutic uses thereof.

### BACKGROUND OF THE INVENTION

The gastrointestinal tract comprises a useful structure conducive to its digestive and absorptive functions, and it also provides a source of highly proliferating cells. The substantial surface area required for the digestive and absorptive functions of the small intestine is achieved by a dense array of tongue-like projections known as villi. The crypts of Lieberkühn are pit-like structures that surround the bases of the villi. In the mouse jejunum, each villus is surrounded on average by 10 crypts (Cheng and Bjerknes, 1985; Cosentino et al., 1996). Cell proliferation is confined to the lower two-thirds of the crypt and, after exiting the cell cycle, most cells migrate out of the crypts onto the villi (Potten and Loeffler, 1990). At least three principal cell lineages are found on the villi, namely: a) absorptive cells (also called enterocytes) that are by far the dominant lineage, making up more than 90% of total cells on the villus; b) goblet cells (also called mucous cells) comprising 8-10% of the villus population; and c) enteroendocrine cells (a diverse group), comprising on average only approximately 1% of the epithelium (Cheng and Leblond, 1974; Wright, 2000). A fourth lineage, namely the Paneth cells, arise from downward migration and are found at the very base of the crypts (Cheng and Leblond, 1974; Wright, 2000).

Pioneering studies by Cheng and Leblond in the early 1970s suggested that undifferentiated cells (which they referred to as “crypt-base columnar cells”) located in the intestinal crypts just above the Paneth cells may serve as multipotent stem cells responsible for the generation of all four major lineages of the small intestinal epithelium. The Unitarian Hypothesis, as it was called (Cheng and Leblond, 1974), has stood the test of time. As reviewed in detail by Gordon, et al. (1992), in the mid-'80s, supporting evidence came from studies of mice chimeras in which the parental origin of intestinal epithelial cells could be identified by lectin staining. These studies showed that in adult mice, whilst crypts are monoclonal, villi can be polyclonal (if supplied by crypts of differing parental origin). Polyclonal villi display coherent ribbons of lectin-stained cells representing multiple lineages. Although these findings with chimeras were consistent with the Unitarian Hypothesis, they did not provide independent evidence pertinent to the multipotency of stem cells in the adult intestine because of the fact that chimeras were generated during the embryonic period. More recently, however, supporting evidence has been forthcoming from studies in which adult mice, with intestinal epithelium that normally stains negative for the lectin Dolichos biflorus agglutinin (Dbl), are subjected to mutagenesis (Bjerknes and Cheng, 1999). This leads to the appearance of lectin-positive ribbons of cells, some of which persist for very long periods of time (greater than 150 days) and thus presumably reflect mutagenesis of a crypt stem cell. The latter ribbons were found to include all four lineages, thus showing conclusively that all arise from a common stem cell (Bjerknes and Cheng, 1999).

The precise number of true stem cells in each crypt remains a matter of some debate and is probably related primarily to the methodology employed. Most approaches consider the number to be 4-6 (Potten and Loeffler, 1990; Bjerknes and Cheng, 1999; Potten, 1998; Winton, 2001), although some studies point to the number being as low as one stem cell per crypt (Cosentino et al., 1996; Gordon et al., 1992). Kinetic data (derived from ³H-thymidine labeling) predict that stem cells are slowly cycling (with cell-cycle times in the order of 24-30 h), in contrast to the proliferative cells in higher positions (and known as transit cells), which typically have a cycle time of 12 h (Potten and Loeffler, 1990; Potten, 1998). Since the total numbers of epithelial cells lining the crypts and covering the villi are well-documented and given the ratio of 10 crypts to each villus, it can be calculated that if there are 1-4 stem cells per crypt, their proportion in the whole epithelium is in the range 0.2-0.8%. The transit cells are normally destined to stop proliferating after 4-6 rounds of cell division at the same time as initiating the process of differentiation and migrating out of the crypts onto the villi (Potten and Loeffler, 1990; Wright, 2000). However, under conditions of insult to the stem cell zone (e.g. by radiation or by cytotoxic drugs) a portion of the transit cells can be called upon to function as stem cells. These are known as clonogenic cells and are critical to the ability of the intestinal epithelium to recover from radiation- and drug-induced injury. Current estimates indicate there are approximately 30 clonogenic cells in each crypt (Potten, 1998).

A critical issue in the study of intestinal epithelial stem cells (IESCs) has been the lack of markers for these cells. Numerous genes are expressed in the crypt compartment but not in the villus (Olsen et al., 2004) and some, such as EphB2 (Batlle et al., 2002) and CD44 (Wielenga et al., 1999) display a gradient of increased expression toward the stem cell zone. However until very recently no protein or mRNA had been observed to be expressed specifically in IESCs. In early 2003, two groups reported, by immunohistochemistry and in situ hybridization, that expression of the RNA binding protein Musashi-1 (Msi-1) is confined to the stem cell zone of the intestinal epithelium (Potten et al., 2003; Kayahara et al., 2003). This protein, which is known to play a key role in asymmetric cell division by neural stem cells (Sakakibara et al., 1996), has thus become a valuable marker for IESCs.

Although the description above constitutes a brief review of the salient aspects of the current knowledge regarding intestinal stem cells, the level of detailed knowledge in this field is illustrated by a great number of recent reviews (Wright, 2000; Gordon et al., 1992; Potten, 1998; Winton, 2001; Booth and Potten, 2000; Clatworthy and Subramanian, 2001).

References in the art considered in accordance with the present invention include the following. Tait et al. (1994) demonstrated that crypt cell aggregates prepared from small intestine of postnatal rats could be transplanted into an isolated loop of proximal colon that had been subjected to surgical mucosectomy. A “neomucosa” with small intestinal morphology and all four epithelial lineages of the small intestine was generated in the majority of recipients.

Slorach et al. (1999) describe an in vivo mouse model for intestinal stem cell function wherein intestinal epithelial cell aggregates (organoids) generate a differentiated small intestinal mucosa having crypt-villus structure. The organoids were obtained essentially as described by Evans et al. (1992), and it is noteworthy that small fragments of the intestine and single cells were removed from the organoids to produce a homogenously sized organoid preparation. Upon engraftment of the organoids, two types of epithelial lined structures are generated, with one being derived from transit amplifying cells and the other from epithelial stem cells. The authors demonstrated that crypt morphogenesis relies on mesenchymal-epithelial interactions in the model.

Booth et al. (1999) determined that adult crypts either freshly isolated or after culture are capable of proliferating, differentiating, and recapitulating morphology at an ectopic site, thus indicating that there are stem cells comprised in these crypts, which are comprised of a monolayer of multiple cells.

U.S. patent application Publication US 2004/0058392 regards stem cell markers for isolation of intestinal stem cells and their use in therapeutic compositions. Methods for isolating the intestinal stem cells comprise providing antibodies against one or more of the target genes, providing single cell suspensions of intestinal tissue, contacting the antibodies and the single cell suspensions, and identifying the cells binding to the antibodies, for example. However, methods for generating single cell suspensions and examples of actual isolations using such antibodies are not provided therein. The application contends that expression of genes associated with TCF/β-catenin is considered to be indicative of stem cell phenotype. For example, TCF target genes listed therein are purportedly definitive for stem cell phenotype of the gut, including CD44, c-Kit, EPHB2, EPHB3, and ENC1, among others. However, published immunohistochemistry indicates these markers are not confined to the stem cells but are also expressed by other cells in the crypt (see, for example, Batlle et al., 2002; Wielenga et al., 1999).

Despite the present knowledge in the art, there are currently no published methods for the isolation of the intestinal stem cells of the present invention. Thus, the present invention provides novel methods and compositions regarding these intestinal stem cells, particularly for therapies related thereto.

### BRIEF SUMMARY OF THE INVENTION

The present invention is generally directed to a system and method that relate to gastrointestinal stem cells, their isolation, and their uses. In particular embodiments, the gastrointestinal stem cells are further defined as or may be otherwise referred to intestinal epithelial stem cells (IESCs). In specific embodiments, the cells are isolated and comprise particular cellular markers.

Amongst the mammalian tissues that display continuous cell turnover (bone marrow, skin and gastro-intestinal epithelium, for example), the epithelium of the small intestine has by far the highest rate of turnover. In both rodents and humans, the majority of cells within the epithelium are replaced every 3-4 days, although certain cell types exhibit slower turnover. As such, the present invention exploits the nature of these highly proliferative cells in contemplating their use for therapeutic purposes.

The four principal cell lineages found in the epithelium (enterocytes, goblet cells, Paneth cells and enteroendocrine cells) are all known to arise from stem cells located deep in the crypts. Despite extensive studies on the kinetics of intestinal stem cells, in both physiological and pathological (e.g., after radiation injury) states, to date there are no published methods for isolation of the gastrointestinal stem cells of the present invention. A related issue is that for any preparation of putative stem cells, characterization of their “stemness” comprises demonstration of the capacity for extensive proliferation and/or the capacity for the production of differentiated progeny. Even cell aggregates (that include epithelial stem cells as well as other cells, including mesenchymal cells, for example) have so far proven refractory to differentiation in vitro. In contrast, such aggregates grafted either subcutaneously or mucosally have been demonstrated to differentiate into all four principal cell lineages of the small intestinal epithelium. Thus, in vivo cell transplant models provide, in some embodiments, conditions for demonstrating “sternness”. In addition, such models are preferable to characterize the desired therapeutic potential of preparations of intestinal epithelial stem cells described herein.

In a specific embodiment of the present invention, intestinal epithelial stem cells share certain characteristics with bone marrow (and other) stem cells through their capacity to be identified and isolated by established flow cytometric methods. Flow cytometric methods are described herein for isolation of viable stem cells from the epithelium of the intestine, such as the exemplary mouse small intestine. For example, intestinal epithelial cells are subjected to sorting procedures that have been successfully used with bone marrow cells to identify a side population (“SP”) fraction that is highly enriched in stem cells. The SP cells are then analyzed for the presence of markers of “stemness” and/or absence of differentiation markers. An additional or alternative sorting approach may utilize assessment of one or more markers with antibodies. Pursuant to these embodiments, a mouse model having local, limited sterilization of intestinal crypts is described such that an ideal denuded region is generated for subsequent transplantation of putative epithelial stem cells.

In other specific embodiments, the potential of intestinal epithelial stem cells to differentiate is related to, such as is dependent upon, specific mesenchymal interactions, and thus transplantation into the small intestine is desirable to examine survival and differentiation; furthermore, such transplantation is favored by conditions that eliminate endogenous stem cells. For example, the present invention encompasses transplantation of intestinal epithelial stem cells into denuded regions of mouse small intestine and subsequent assessment of the morphology and/or the expression of lineage-specific markers in epithelium derived from the transplanted cells. In particular embodiments, to facilitate identification the donor cells are from ROSA 26 mice and, thus, are tagged, such as by expression of bacterial β-galactosidase, for example.

In an object of the invention, there is an isolated mammalian gastrointestinal stem cell, wherein the cell is characterized as the following: CD45 negative; Collagen IV negative; and Msi-1 positive. In specific embodiments, the cell may also comprise one or more characteristics selected from the group consisting of Hes-1 positive, CD34 negative, Thy1.2 negative, Sca1 negative, c-kit negative, EphB2 positive, CD44 positive, Math-1 positive, Hath-1 positive, and Brcp1 positive. Cells of the invention may be isolated from the gastrointestinal tract, including the stomach, large intestine, or small intestine. Therefore, the cell may be isolated from the stomach, duodenum, jejunum, ileum, colon, cecum, or rectum. In particular embodiments, the cell is isolated from the jejunum, the ileum, and/or the colon. The cell may be isolated from a mammal of any age, including an adult or infant, and the mammal may be any kind of mammal, including a human, mouse, or rat, for example. In particular embodiments, mammals receiving treatment with the present invention may be the same species from which the isolated cells are obtained or the mammal receiving treatment may be from a different species from which the isolated cells are obtained.

In particular embodiments, the cell is further defined as being in a suspension of cells that are substantially single in nature. The suspension of substantially single cells can be further defined as being substantially free of organoids. In another particular embodiment, the suspension of substantially single cells is suitable for a cell sorting method. The suspension of substantially single cells is obtained, for example, from the method of providing gastrointestinal tissue from a mammal; subjecting the tissue to mechanical disruption, chemical disruption, enzymatic digestion, or a combination of two or more thereof, wherein said subjecting step generates the suspension of substantially single cells.

Mechanical disruption of intestinal tissue for methods described herein comprises cutting, chopping, slicing, shaking, or shearing the gastrointestinal tissue, for example. Chemical disruption comprises, for example, disruption with EDTA, mannitol, or a mixture thereof. Enzymatic digestion comprises, for example, dispase, collagenase, trypsin, pancreatin or other proteases, or a mixture thereof, and the digestion duration may be greater than about 25 minutes, in some embodiments. Isolation methods described herein may also further comprise the step of removing mucus from the gastrointestinal tissue.

In another object of the present invention, there is an isolated mammalian gastrointestinal stem cell that is CD45 negative; Collagen IV negative; and Msi-1 positive, wherein the cell is obtained from the method of providing gastrointestinal tissue from a mammal; and subjecting the tissue to mechanical disruption, chemical disruption, enzymatic digestion, or a combination of two or more thereof, wherein the subjecting step generates the isolated stem cell.

In an additional object of the invention, there is a method of isolating a mammalian gastrointestinal stem cell, comprising the steps of providing gastrointestinal tissue from the mammal; and subjecting the intestinal tissue to at least one disruption method, wherein the subjecting step generates a plurality of single cells, said plurality of single cells defined as substantially free of cell aggregates, wherein the single cells are defined as being: CD45 negative; Collagen IV negative; and Msi-1 positive. The providing step comprises removing tissue from the jejunum of the mammal, for example.

In another object of the invention, there is a method of treating intestinal tissue in an individual in need thereof, comprising obtaining isolated stem cells comprising the following characteristics: CD45 negative; Collagen IV negative; and Msi-1 positive; and delivering the stem cells to the intestine in need of treatment. The intestine of the individual in need of therapy can be due to necrotizing enterocolitis, infarction, injury, insulin-dependent diabetes, cancer, hormone deficiency; clotting disorder, phenylketonuria, a metabolic disorder, inflammatory bowel disease (IBD), or ulcer, for example. The hormone deficiency comprises deficiency of growth hormone and the metabolic disorder may be a urea cycle disorder, in specific embodiments.

In particular aspects of the invention, the individual being administered therapy of the present invention was treated and/or is being treated with therapy that is damaging to the intestine, such as cancer therapy or therapy with at least one nonsteroidal anti-inflammatory drug. The cancer therapy is chemotherapy, radiation, surgery, or a combination of two or more thereof, in some embodiments. The isolated stem cells are delivered to the intestine in need thereof prior to the therapy that is damaging to the intestine, concomitant with the therapy that is damaging to the intestine and/or subsequent to the therapy that is damaging to the intestine. The stem cells may be isolated from the individual in need thereof and/or from an individual which is not the individual in need thereof.

In another object of the invention, there is a method of treating intestinal tissue in an individual in need thereof, comprising obtaining at least one isolated stem cell comprising the following characteristics: CD45 negative; Collagen IV negative; and Msi-1 positive; and introducing to the stem cell a polynucleotide encoding a gene product that is therapeutic for the intestinal tissue; and delivering the stem cell comprising the polynucleotide to the intestine in need of treatment. The stem cells are isolated from the individual in need thereof or from an individual which is not the individual in need thereof.

An individual in need thereof may be any kind of animal, but in particular embodiments the individual is a mammal, such as a human. In specific embodiments, there is interspecies delivery of the isolated stem cells. For example, stem cells of the invention may be obtained from one species and delivered to an individual of another species, such as the cells being isolated from a pig, mouse, goat, rabbit, rat, dog, cat, monkey, chimpanzee, or gorilla and delivered to a human.

In an additional object of the invention, there is a method for preventing, treating, or both, intestinal damage in an individual with cancer, comprising administering radiation, chemotherapy, or both, to the individual; obtaining at least one isolated mammalian gastrointestinal stem cell comprising the following characteristics: CD45-negative; collagen IV-negative; and Msi-1-positive; and delivering the at least one isolated stem cell to the intestine of the individual. The stem cell can be delivered to the individual prior to administration of the radiation and/or chemotherapy step, concomitant with administration of the radiation and/or chemotherapy step, and/or subsequent to administration of the radiation and/or chemotherapy step. The stem cell can be obtained from the individual prior to the administering of the radiation and/or chemotherapy step and/or from another individual or individuals that are not the individual with cancer.

In an additional object of the invention, there is a cancer treatment method comprising administering cancer therapy, such as chemotherapy and/or radiation, into an individual with cancer, obtaining isolated stem cells characterized as being CD45 negative; Collagen IV negative; and Msi-1 positive, and delivering the stem cells to the individual. In particular embodiments, the stem cells are obtained from the individual with cancer prior to administration of the cancer therapy and/or they may be obtained from another individual or individuals that are not the individual with cancer.

In specific embodiments, there is a method of testing whether one or more cells has intestinal stem cell activity, comprising the steps of providing a mammalian model comprising one or more of the following intestinal characteristics: having at least reduced numbers of endogenous intestinal stem cells (compared with normal); having intact basement membrane; and having intact mesenchyme; delivering one or more cells to be tested to the intestine of the model; and assaying the model for engraftment, differentiation, or both of said test cells, wherein when said test cells engraft, differentiate, or both, said test cells are stem cells that are CD45 negative, Collagen IV negative; and Msi-1 positive.

The providing step can be further defined as ablating some or all of the endogenous intestinal stem cells, which may be done in any manner including administering an agent to the endogenous intestinal stem cells, such as by administering the agent luminally or systemically, including by intraperitoneal administration. The agent may be capable of local action upon a region of the intestine, and the agent may provide no detectable damage to the basement membrane, stromal tissue, or both. In specific embodiments, the agent is doxorubicin. In an alternative embodiment, radiation is employed to ablate some or all of the endogenous intestinal stem cells. The radiation may be applied to the whole body of the individual or a portion of the intestine of the individual (such as in the form of a pellet, for example).

The providing step can be further defined as segregating a region of the intestine from the remainder of the intestine; and delivering the agent to the segregated region, such as through clamping at least part of the jejunum. One or more of the test cells can be delivered luminally or systemically.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying exemplary drawings.

FIG. 1 provides FACS analysis of adult mouse jejunum.

FIG. 2A shows qRT-PCR for Msi-1 mRNA in cell fractions vs. intact intestine (n=3). FIG. 2B shows Msi-1 protein (brown) is localized to stem cell zone. (from Kayahara et al. (2003)).

FIG. 3 provides data showing apoptosis after luminal delivery in clamped segments of the intestine.

FIG. 4 shows the position of apoptosis after exposure to luminally administered doxorubicon (DOX).

FIG. 5 illustrates that the crypt niche remains intact after exposure to luminally administered DOX.

FIG. 6 shows a time course of apoptosis after IP DOX at 20 mg/kg body weight (BW).

FIG. 7 demonstrates morphologic damage in mouse jejunum before and 96 hours after IP DOX (20 mg/kg BW).

FIG. 8 shows qRT-PCR for Msi-1 and for markers of stromal cells and differentiated epithelial lineages in CD45-negative SP from mouse jejunum as compared with intact jejunal tissue. Hatched bars show mean±SE (n=3) for Msi-1, sucrase-isomaltase (SI), trefoil factor 3 (TFF3), and lysozyme mRNA. The solid bar represents the intact tissue level of each marker.

FIG. 9 illustrates an exemplary temporarily bypassed loop. Dotted line shows anastomosis and arrow shows position of temporary obstruction.

FIG. 10 demonstrates X-gal staining of experimental segment from wildtype mice 21 days after transplantation with jejunal cell aggregates from ROSA 26 mice. ROSA epithelium shows as blue stain.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Also, commensurate with well-established patent law, the term “comprising” hereby refers to “comprising at least”.

I. Definitions

The term “epithelial” as used herein relates to the lining of the internal cavity of the intestine. The intestinal epithelial cells are joined by structures known as tight junctions that provide adhesion between the cells that is relatively difficult to break apart. This reflects the fact that one of the important functions of this epithelium is to serve as a barrier.

The term “gastrointestinal” as used herein refers to the stomach, large intestine, and small intestine.

The term “infant” as used herein refers to a very young mammal of the age wherein sustenance is available by suckling a teat of a mother.

The term “intestinal epithelial cell” as used herein refers to a cell from the epithelium of the gastrointestinal tract.

The term “intestinal” as used herein pertains to the intestine, which comprises both the large intestine (also referred to as the colon) and the small intestine. The small intestine is comprised of at least three sections: duodenum, jejunum and ileum. All sections of the small intestine are involved in the absorption of nutrients. The large intestine comprises at least the caecum, ascending colon, transverse colon, descending colon, sigmoid colon and rectum. The large intestine is responsible for forming, storing and expelling waste matter.

The term “mesenchymal” as used herein refers to tissue of mesodermal (the middle of the three germ layers) origin.

The term “negative” as used herein refers to the absence of a particular marker or its expression thereof in a cell under standard conditions. The negative nature of the marker is usually reflected in its expression or presence of the gene product being at undetectable levels in a particular cell, for example. The term “undetectable” as used herein refers to being undetectable according to standard analytical techniques in the art. The term “negative” describes marker expression in a fraction of at least mostly stem cells as being lower in abundance (such as “de-enriched”) compared to expression levels seen in intact intestine. Whether or not a cell is positive or negative for a particular marker may be determined by any suitable means, although, for example, assessment of antibody binding to the respective marker on or in the cell, such as a cell surface marker, is encompassed within the invention, as is the detection of expression of the gene encoding the marker, such as by RNA detection. In a particular aspect, stem cells may be distinguished based not only on the presence or absence of particular markers but on the degree of expression of one or more particular markers.

The term “organoid” as used herein refers to those cell aggregate structures, such as from known intestinal tissue processing methods that comprise epithelium (such as about 90% of the organoid) with attached mesenchyme (such as about 10% of the organoid). The organoids have high viability yet are not amenable to subsequent manipulations, such as those suitable for a plurality of single cells. In particular embodiments, organoids may comprise stem cells, although as they are cell aggregates, they are not comprised of single stem cells in suspensions. The term “aggregate” as used herein refers to cells massed into a cluster. In a specific embodiment, at least the majority of the cells in the aggregate are physically adhered together. In a specific embodiment, the isolated stem cell of the present invention is not isolated as part of an organoid, such as is described in Booth et al. (1999), for example, but rather is isolated as a plurality of mostly single cells unhindered by adhesion to other cells.

The term “positive” as used herein refers to the presence of a particular marker or its expression thereof in a cell, such as under standard conditions. The positive nature of the marker is usually reflected in its expression or presence of the gene product being at detectable levels in a particular cell, for example. Whether or not a cell is positive or negative for a particular marker may be determined by any suitable means, although detection of antibody binding to the respective marker, such as a cell surface marker, is encompassed within the invention, as is the detection of expression of the gene encoding the marker, such as by RNA detection. The level of expression may also be considered in accordance with cell markers.

The term “side population (SP)” as used herein refers to a small fraction of cells that can be separated from the majority of the cells based on their behavior when subjected to fluorescence activated cell sorting (FACS) following staining of the cell suspension with the DNA-binding dye Hoechst 33342, wherein the behavior is characteristic of stem cells. In alternative embodiments, another suitable DNA-binding dye is utilized, such as one that is not toxic. In a specific embodiment, rhodamine is employed as the DNA-binding dye. In some embodiments, some subfraction of the SP cells may have greater stem cell potency.

The term “stem cell” as used herein refers to a cell that gives rise to one or more lineages of cells. In specific embodiments, stem cells preferably comprise the ability to maintain a large proliferative capacity and produce most or all epithelial lineages of the small intestine. In particular embodiments, the stem cells of the invention facilitate, enhance or otherwise make possible the ability to regenerate crypt-villus structural integrity upon delivery to an appropriate intestinal environment.

The term “substantially free of mesenchymal tissue, mesenchymal cells, or both” as used herein refers to a suspension of cells wherein the majority of cells therein are not themselves nor in contact with mesenchymal tissue, cells, or both, respectively. In specific embodiments, the substantially free cells are considered isolated and/or purified, and greater than about 90% of the isolated and/or purified cells may not be in contact with mesenchymal tissue, cells, or both. In alternative embodiments, the cells may be with mesenchymal tissue, mesenchymal cells, or both but are suitable so long as they are engraftable.

The term “substantially free of organoids” as used herein refers to a suspension of cells wherein the majority of cells therein are not in clusters known as an organoid. In a specific embodiment, about 95% of the cells are not in organoids.

The term “suspension of substantially single cells” as used herein refers to a plurality of single cells wherein the majority of cells are not comprised in a cellular aggregate. The term may be further defined as the majority of cells within the suspension being free of adhesion to one or more cells. In a specific embodiment, the cells are isolated and purified, and no more than 10% of the cells are mesenchymal.

The term “therapeutically effective” as used herein refers to an amount of cells and/or therapeutic composition (such as a therapeutic polynucleotide and/or therapeutic polypeptide) that is employed in methods of the present invention to achieve a therapeutic effect, such as wherein at least one symptom of a condition being treated is at least ameliorated.

II. The Present Invention

The present invention provides a novel isolated gastrointestinal stem cell comprising several defining characteristics, such as cell markers, the methods of isolating the novel cells, and therapeutic uses thereof. In particular embodiments, the intestinal stem cell is CD45 negative, collagen IV negative, and Msi-1 positive. The cell is preferentially comprised in a suspension of substantially single cells, as opposed to being in cell aggregates including organoids known in the art. As such, the suspension of cells may be considered substantially organoid-free.

Although Msi-1 positive intestinal stem cells have been identified (Kayahara et al., 2003), these cells were not isolated, and the present invention provides additional guidance as to both 1) specific marker(s) characterisitc of intestinal epithelial stem cells and, furthermore; and 2) optimal means to obtain these better-identified cells as isolated entities, as opposed to being comprised in a cell aggregate. The nature of having a suspension of single cells generated from intact intestinal tissue or tissue fragments thereof facilitates their utilization in subsequent methods, such as flow cytometric methods that generate the characteristic side population of stem cells from a cell mixture following exposure to Hoechst 33342 dye, as demonstrated by the inventor.

Prior to the invention, intestinal stem cells were considered unviable in culture. Indeed, the goal of Evans et al. (1992) was to generate isolated cells in culture, but the cells would not proliferate. In fact, Evans et al. concluded that proliferation of gut epithelium ex vivo requires preserving the structural integrity of the tissue and requires factors produced by underlying mesenchymal cells. It was considered to be critical to in vitro survival for the crypt units to have the underlying mesenchymal cells (such as myofibroblasts) to still be attached. Contrary to the art, however, the inventor successfully isolated cells that are not only amenable to subsequent manipulations but that also remain viable. Furthermore, the isolated cells of the invention may be comprised in a composition suitable for a therapeutic purpose, such as replenishing cells in an intestine or intestinal region in need thereof.

Further, the availability of substantially pure populations of intestinal epithelial stem cells (IESCs), such as the exemplary mouse IESCs, allows the following, for instance: a) microarray studies to characterize patterns of gene expression in these cells to characterize the nature of sternness; b) in vitro studies to find conditions that would allow both proliferation (and thus expansion for therapeutic purposes) and/or differentiation; and/or c) characterization of their ability to rejuvenate intestinal epithelium in diseased and/or damaged intestine. In particular, cell separation methods of the present invention are applied to human small intestine. Also, the intestinal damage/transplantation model may be utilized to characterize the one or more factors responsible for homing of stem cells to the stem cell niche and to identify one or more local factors important for both proliferation and lineage determination. In addition, models described herein are utilized to characterize intestinal stem cells as vehicles for introducing genes into the intestinal epithelium for the purpose of gene therapy. Finally, the stem cells of the present invention are useful to replenish cells destroyed or impaired during therapy, such as upon chemotherapy and/or radiation therapies, for example.

III. Methods for Isolation of Stem Cells

Bone marrow hematopoietic stem cells (BM-HSC) are by far the most widely studied amongst all mammalian stem cells. The classical approach to the isolation of BM-HSC has been the use of surface markers and their respective antibodies, which have allowed fluorescence activated cell sorting (FACS), also known as flow cytometry (Goodell et al., 1997; Zhou et al., 2001). A novel method for isolation of BM-HSCs was discovered by Goodell, et al. (Jackson et al., 1999) following the staining of bone marrow cells with the fluorescent DNA-binding dye Hoechst 33342. These authors noticed that when the stained population was subjected to dual wavelength flow cytometry, a small side population (SP) of cells became apparent. When the ability of SP cells to repopulate bone marrow of lethally irradiated host mice was examined, it was found that the SP fraction is enriched more than 1000 times compared with unfractionated bone marrow (Jackson et al., 1999). Comparison of the SP from murine bone marrow with classical separation methods based on surface markers demonstrates that the SP represents a purer preparation of HSCs (Gussoni et al., 1999). Subsequently, the SP phenotype has been shown to characterize stem cells of skeletal muscle (Wulf et al., 2001; Orkin, 2000; Quaroni and Hochman, 1996), mammary gland (Kedinger et al., 1987; Bens et al., 1996) and liver (Whitehead et al., 1993). For these tissues, cell transplantation experiments (with appropriately tagged donor cells) have shown that the respective SP cells can contribute to tissue regeneration. Orkin (2000) has expressed the view that the SP phenotype is more likely than any of the established surface markers to be a shared characteristic of stem cells from diverse tissues. The present inventor demonstrates herein that cell suspensions generated from mouse intestinal epithelium display an SP phenotype and that intestinal SP cells are greatly enriched for Msi-1 expression. Thus, the present inventor has successfully isolated a fraction of viable cells that comprises the IESCs.

IV. Mucosal Transplantation Models

To date there is only one report of transplantation of intestinal epithelial cells directly into the intestinal mucosa. In this study, Tait, et al. (1994) showed that crypt cell aggregates prepared from small intestine of postnatal rats could be transplanted into an isolated loop of proximal colon that had been subjected to surgical mucosectomy. A “neomucosa” with small intestinal morphology and all four epithelial lineages of the small intestine was generated in 76% of recipients. Others have used artificial membranes on which are seeded either epithelial cells (Kawaguchi et al., 1998) or cell aggregates (Kaihara et al., 1999; Kaihara et al., 2000). Such preparations can be anastamosed to the native intestine and subsequently show both survival and proliferation of the introduced epithelial cells (Kawaguchi et al., 1998; Kaihara et al., 2000). As expected from the findings with ectopic transplantation, epithelial cells alone remained as an undifferentiated monolayer (Kawaguchi et al., 1998), whereas aggregates (which included mesenchymal cells) yielded a neomucosa with relatively normal crypt/villus morphology (Kaihara et al., 2000). The latter appears to be dependent on exposure to luminal contents as non-anastomosed tissue shows only rudimentary morphology (Tavakkolizadeh et al., 2003). While both of these models (mucosectomy and artificial membranes) have merit, neither is considered ideal for the assessment of potency of preparations of putative IESCs, because both are very labor-intensive (requiring microsurgery), thus limiting the number of preparations and conditions that can be tested.

V. Models of Crypt Damage

Two simple surgical models have been developed in which putative IESCs can be introduced into a region of small intestine that has been partially denuded of endogenous stem cells but which has the basement membrane, underlying mesenchymal structures and other elements of the stem cell niche (Mills and Gordon, 2001; Paris et al., 2001; Bjerknes and Cheng, 2001) remaining intact. Both of these models are based on the use of the chemotherapeutic agent doxorubicin (DOX), also known as Adriamycin, and they are described at least in Examples 7 and 9. In the first model, DOX is delivered luminally to a clamped segment of small intestine for 4-6 hr, thus causing local crypt damage. Preparations of putative IESCs can then be delivered intravenously. In specific embodiments, signals arising from the damaged region of the intestine cause the circulating IESCs to home to that region and engraft in the damaged crypts. The second model takes a converse approach, namely global crypt damage by intraperitoneal delivery of DOX followed by local luminal delivery of putative IESCs into clamped segments. These models are a favorable environment for engraftment and differentiation of putative IESCs, i.e. an ideal opportunity to study stemness of our SP cells. These models also allow characterization of the embodiment wherein transplantation of SP cells afford functional restitution of the epithelium following crypt damage, and thus study of the therapeutic potential of IESCs is facilitated.

VI. Colonic Stem Cells

As in the small intestine, multipotent colonic epithelial stem cells are located deep in the colonic crypts (Wright, 2000; Winton, 2001; Yatabe et al., 2001; Chang and Leblond, 1971). Understanding both normal and aberrant behavior of colonic stem cells is critical to the field of colon cancer (Wright, 2000; Winton, 2001; Wright, 2000; Booth et al., 2002). Moreover, the availability of colonic stem cells in some embodiments provides novel approaches to the restitution of damaged bowel. Thus, a method to isolate these cells is useful. To date, the only published attempts are those from the laboratory of R. H. Whitehead (Fujimoto et al., 2002; Whitehead et al., 1999). Although these studies have constituted a major advance in the field (being the first to demonstrate clonogenic growth in vitro), they have not yielded a fraction that is highly enriched with colonic stem cells. In a particular aspect of the invention, just as in the small intestine and other tissues, colonic epithelial stem cells can be isolated by SP sorting following staining with Hoechst 33342. As Msi-1 has also been reported to be a marker of colonic stem cells (Potten et al., 2003; Nishimura et al., 2003), after generating SP cells from the colonic epithelium in specific embodiments they are further identified as colonic stem cells via assessment for Msi-1 mRNA, such as by quantitative RT-PCR described herein.

VII. Gene Expression Profiling of Stem Cell Populations

There currently is no known common set of expressed genes in all stem cell preparations. However, the Stem Cell Genome Anatomy Project will facilitate the characterization of stem cell populations by providing open communication for a variety of labs to exchange information for their respective systems. Furthermore, microarray profiling of the inventive preparations of IESCs may be employed in the present invention to elucidate markers additional to those exemplary markers described herein. In specific embodiments, gene expression profiling of putative IESCs obtained by SP sorting as compared with putative IESCs obtained by laser capture microdissection allows identification of a subset of transcripts that are strong candidates for being true IESC transcripts.

VIII. Therapeutic Applications

With respect to ultimate therapeutic potential, the ability to rejuvenate the small intestinal and colonic epithelium by stem cell transplantation is useful for application in pathological conditions of the bowel. In this regard, it should be noted that despite attempts over more than 10 years, small bowel transplantation remains limited to few centers around the world and to select patients with life-threatening complications of Total Parenteral Nutrition (TPN) (Gilroy and Sudan, 2000; Kaufman et al., 2001). For patients with intestinal failure, for example, therapeutic options are currently very limited. Human IESCs are useful for both autologous and heterologous transplantation. In agreement with the “Roadmap to the Clinic” in a recent review of somatic (adult) stem cells (Daley et al., 2003), in a specific embodiment of the invention disorders directly affecting the IESCs themselves are likely to be the most amenable to stem cell therapy. Radiation/chemotherapy-induced enteritis is one specific embodiment. Ulcers or other injury due to either injectious agents, chemical agents or ischemia are other examples. Additional examples of intestinal failure with more complex pathologies (e.g. necrotizing enterocolotis, inflammatory bowel disease, infarction and injury due to trauma) are also amenable to stem cell therapy, in particular aspects of the invention. Thus, in particular embodiments the isolated stem cells themselves as therapeutic compositions are delivered to an intestine in need thereof. Delivery of the isolated cells to the intestine may be accomplished by any method suitable to deliver enough cells to the intestinal region in need thereof such that at least one symptom of the defective, injured, diseased or otherwise unhealthy intestine is at least ameliorated.

A further application of the present invention comprises seeding of intestinal stem cells onto an appropriate artificial membrane together with appropriate mesenchymal cells in order to generate a patch or a tube of artificial intestine for use in extreme cases of intestinal failure, injury and damage as described above. Although such tissue engineering has been performed with organoids (Kawaguchi et al., 1998; Kaihara et al., 1999; Kaihara et al., 2000, the present invention encompasses the distinct advantage of engineering intestine utilizing isolated stem cells of the present invention, particularly through providing a suspension of substantially single cells from which the tissue is ultimately engineered.

Furthermore, the present inventor and others have noted previously that the epithelium constitutes an ideal site for gene therapy approaches both to intestinal disorders and to a variety of genetic, metabolic and endocrine disorders of other tissues (Foreman et al., 1998; Henning, 1995; Sferra et al., 1997; Forbes and Hodgson, 1997; Cheng et al., 1997). Thus, in addition to or in lieu of delivering isolated stem cells themselves as therapeutic compositions for the intestine, the inventive stem cells may further comprise a therapeutic composition, such as a therapeutic polynucleotide and/or a therapeutic polypeptide. The therapeutic gene may be present endogenously within the cell or it may be introduced into the cell prior to delivery of the cell to the intestine. Introduction of nucleic acids into cells are well known in the art and may be accomplished by, for example, calcium phosphate and/or DEAE-dextran methods, electroporation, particle bombardment, direct microinjection, lipid emulsions, viral vectors and/or sonication loading, and so forth. The expression of the therapeutic polynucleotide is preferably controlled by sequences operable in an intestinal stem cell and/or in one of the differentiated intestinal lineages.

In another embodiment, the inventive isolated intestinal stem cells are delivered themselves as therapeutic compositions to the intestine in need thereof, and a therapeutic polynucleotide is delivered to these newly introduced stem cells thereafter. Delivery of the therapeutic polynucleotide into the intestine having the newly introduced stem cells may employ any suitable method, and in a particular embodiment the therapeutic polynucleotide is introduced into the intestine by such methods described in U.S. Pat. No. 5,786,340 and U.S. Pat. No. 5,821,235, both of which are incorporated by reference herein in their entirety. In the methods of U.S. Pat. No. 5,786,340, a section of an intestine is ligated or clamped such that it forms a closed cavity, and a vector solution comprising the therapeutic polynucleotide is injected into the closed cavity such that the intestine is distended, followed by removal of the ligation or clamp. In the methods of U.S. Pat. No. 5,821,235, an insertion device is introduced into a section of the intestine, a vector solution comprising a therapeutic polynucleotide is introduced via the insertion device such that the cells contact the vector solution for a sufficient time to incorporate the therapeutic polynucleotide into the cells. In particular embodiments, the insertion device is a catheter.

Although attempts of direct gene transfer to intestinal epithelial stem cells in vivo have met with limited success (Sferra et al., 1997; Lau et al., 1995; Jacomino et al., 1996), this can be attributed largely to the difficulty of generating high titers of suitable vectors (Henning, 1995; Sferra et al., 1997; Grand et al., 1999). In contrast, in vitro gene transfer is generally more efficient (Noel et al., 1994; Jacomino et al., 1997) and, thus, IESCs permit ex vivo genetic manipulation prior to transplantation. Therefore, the inventive isolated cells described herein offer an advantage to providing gene therapies to diseased or damaged intestines. Among the disorders that would be amenable to such a gene therapy approach include insulin-dependent diabetes, wherein the polynucleotide would encode insulin; growth hormone deficiency, wherein the polynucleotide would encode growth hormone; other deficiencies of circulating protein hormones, wherein the polynucleotide would encode the respective hormone; other disorders of circulating proteins, such as clotting disorders, wherein the polynucleotide would encode the relevant clotting factor; phenylketonuria, wherein the polynucleotide would encode phenylalanine hydroxylase; urea cycle disorders, wherein the polynucleotide would encode the missing enzyme; other metabolic disorders, wherein the polynucleotide would encode the missing enzyme; inflammatory bowel disease (IBD), wherein the polynucleotide would encode an anti-inflammatory cytokine or an inhibitor of inflammatory cytokines; other intestinal disorders, wherein the polynucleotide would encode the relevant missing enzyme; tumors of the gastrointestinal tract, wherein the polynucleotide would encode a secreted product with anti-tumor activity; and/or damage to the gastrointestinal tract (such as ulcers, necrotizing enterocolitis, radiation or chemotherapy-induced enteritis), in which case the polynucleotide(s) would encode one or more factors known to enhance rejuvenation of the epithelium (e.g. prostaglandins, EGF, FGF, GLP-2, etc.). Insulin-dependent diabetes in a preferred aspect of the invention is treatable with compositions and methods of the present invention, particularly given that physiologically-appropriate, glucose-regulated insulin secretion can be achieved in enteroendocrine cells of the intestinal epithelium (Cheung et al., 2000).

Recent studies have pointed to an abundance of new players that in some aspects of the invention are important in the biology of stem cells of the GI tract. The list includes: transcription factors produced within epithelial cells, such as Tcf-4, β-catenin, cdx-1, HFH11, HNF3, Ihh and Math1 (Wright, 2000; Winton, 2001; Korinek et al., 1998; Ramlho-Santos et al., 2000; Yang et al., 2001); other factors produced in the surrounding mesenchyme, such as Fkh6, Hlx, Nkx2-3, epimorphin and Bmp4 (Wright, 2000; Winton, 2001; Vidrich et al., 2003; Clatworthy and Subramanian, 2001; Kaestner et al., 1997) as well as the tyrosine kinase receptors EphB2 and EphB3, which have been shown to be involved in cell migration and positioning in intestinal crypts (Batlle et al., 2002). Likewise molecules which serve as effectors of differentiation of the epithelium have just begun to be identified (Wright, 2000; Winton, 2001; Clatworthy and Subramanian, 2001; Ye et al., 1997). In specific embodiments of the present invention, polynucleotides encoding these gene products and/or the gene products themselves are delivered to a gastrointestinal tract in need of therapy via the stem cells of the present invention.

The method or methods employed to deliver the inventive intestinal stem cells to an intestinal tissue in need thereof may be of any kind so long as at least some of the cells are delivered such that they provide a therapeutic result. The cells may be applied systemically, luminally, intraperitoneally, or by direct injection into the intestinal mucosa, for example.

IX. Methods of Nucleic Acid Delivery

In some embodiments of the present invention, there is delivery of a therapeutic nucleic acid (which may be referred to as a therapeutic polynucleotide) into a cell of the invention or into an individual receiving a cell of the invention, for example.

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

1. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of nucleic acid used may vary upon the nature of the nucleic acid as well as the organelle, cell, tissue or organism used.

2. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre B lymphocytes have been transfected with human kappa immunoglobulin genes (Potter et al., 1984), and rat hepatocytes have been transfected with the chloramphenicol acetyltransferase gene (Tur Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

4. DEAE Dextran

In another embodiment, a nucleic acid is delivered into a cell using DEAE dextran followed by polyethylene glycol. In this manner, reporter plasmids were introduced into mouse myeloma and erythroleukemia cells (Gopal, 1985).

5. Sonication Loading

Additional embodiments of the present invention include the introduction of a nucleic acid by direct sonic loading. LTK fibroblasts have been transfected with the thymidine kinase gene by sonication loading (Fechheimer et al., 1987), for example.

6. Liposome Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non histone chromosomal proteins (HMG 1) (Kato et al., 1991). In yet further embodiments, a liposome may be complexed or employed in conjunction with both HVJ and HMG 1. In other embodiments, a delivery vehicle may comprise a ligand and a liposome.

7. Receptor-Mediated Transfection

Still further, a nucleic acid may be delivered to a target cell via receptor-mediated delivery vehicles. These take advantage of the selective uptake of macromolecules by receptor mediated endocytosis that will be occurring in a target cell. In view of the cell type specific distribution of various receptors, this delivery method adds another degree of specificity to the present invention.

Certain receptor-mediated gene targeting vehicles comprise a cell receptor specific ligand and a nucleic acid binding agent. Others comprise a cell receptor specific ligand to which the nucleic acid to be delivered has been operatively attached. Several ligands have been used for receptor mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, have been incorporated into liposomes and observed an increase in the uptake of the insulin gene by hepatocytes (Nicolau et al., 1987). It is contemplated that the tissue specific transforming constructs of the present invention can be specifically delivered into a target cell in a similar manner.

8. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Methods to Generate Single Cell Suspensions of Intestinal Tissue Suitable for Flow Cytometry

Although flow cytometry has been used for cell cycle analyses of the intestinal epithelium (Cheng and Bjerknes, 1985; Pallavicini et al., 1984), such studies typically use glutaraldehyde fixed cells (Cheng and Bjerknes, 1983). To date, there are no reports on the use of flow cytometry to separate viable cells from the epithelium (the exception being intraepithelial lymphocytes). Thus, in a specific embodiment of the invention, mucous-free single cell suspensions of mouse intestinal epithelium are produced, and the cells remain viable through the manipulations required for preparation, staining and sorting. In an additional specific aspect of the invention, staining with Hoechst 33342 yields a side population (SP), as in other tissues.

To pursue both these goals, adult mouse intestine was dissociated, such as by a modification of the method of Evans, et al. (1992) and then subjected to differential sedimentation to generate epithelial cell aggregates (Evans et al., 1992). These aggregates were further dissociated, such as by serial filtration (Cheng and Bjerknes, 1985). As clumped cell preparations cannot be used for flow cytometry, all washing and filtration steps in the preparation used high volumes and multiple replications in order to remove mucous. Microscopic examination showed that this approach reproducibly generated single cell suspensions. The cell suspensions were stained with Hoechst 33342 as described by Goodell, et al. (1996; 1997) and co-stained with propidium iodide (PI) to identify dead cells. Following flow cytometry, 51% of the cells were stained by PI, indicating that 49% of the population remained viable. This is within the range observed for successful stem cell preparations from other tissues.

In addition to this method, and bearing modifications of the Evans et al. (1992) procedure, the present inventor developed the following method for isolating single cells from intestinal tissue.

Reagents include Hanks Buffered Saline Solution (HBBS) (Gibco, cat# 24020-117); HBSSplus (HBSS with 5% FBS; Gibco, cat# 26140-079); Dispase (Gibco, cat# 17105-041, (0.78 U/mg)); and Collagenase Type III Fraction A (Sigma, cat# C-0255 (411 U/mg)). The Enzymatic Solution comprises 20 ml HBSS with 6 units of Dispase (0.3 U/ml) and 300 units of Collagenase (15 U/ml). The exemplary method is as follows:

1. Remove the jejunum from two adult mice, flush with HBSS at RT. Split open the jejunum lengthwise. Cut jejunum into small 33mm lengths. Transfer the pieces to a T25 flask. Wash the pieces by shaking for 30 seconds on orbital shaker (80 RPM or setting 20 on New Brunswick platform shaker) with 20 ml HBSS, decant media and repeat process for a total of 5 washes at RT.

2. Transfer the clean pieces to a Plexiglas board and dice into <1 mm sections using a clean razor blade. Return the pieces to a new T25 flask and add the 20 ml of Enzymatic Solution. Shake for 45 min at RT at 80 RPM.

3. Pipette tissue up and down for 15 min using a 10 ml disposable pipette to dissociate cells. Transfer the contents to a 50 ml conical tube. After dissociation of cells, add 1 ml of FBS to make it 5% serum (this will stop the collagenase from tearing up the cells).

4. Let the content sediment under gravity for 1 min. Pipette off the supernatant leaving about 2-3 ml behind (this is the residual). Transfer the supernatant to a new 50 ml conical and allow to sediment 2 more times for total of 3.

5. Centrifuge the supernatant from step 4 at 300 RPM for 3 min.

6. Resuspend pelleted cells in a total of 10 ml of HBSSplus (Gently resuspend cells in 1 ml of solution and then bring up to 10 ml). Pass the cells through a 70-micron mesh sieve, and then count. In specific embodiments, there are about 1.5 to 2 million cells/ml.

This method advantageously yields single cell suspension suitable for cell sorting. In comparison with the Evans et al. (1992) method, there are at least the following exceptions:

1. Dispase: crude 0.3 U/ml vs. pure 0.6 U/ml.

2. Collagenase: pure Type III at 15 U/ml vs. crude Type XI at 300 U/ml.

3. Duration of Digestion: 45 min vs. 25 min

4. HBSSplus is used from step 3 onwards, whereas Evans et al. introduce FBS only at step 7 where DMEM-S is introduced.

The final cell pellet is washed only once, whereas Evans et al. wash 5-6 times.

In another embodiment, an alternative method that provides greater speed of isolation and a purer preparation of epithelial cells as the starting material is the in vivo EDTA method of Bjerknes and Cheng (1981). It involves intracardiac perfusion of adult mice with 30 mM EDTA for 3 min followed by eversion of the intestine onto a glass rod. When the rod is vibrated, the entire epithelium lifts off as a single sheet (Bjerknes and Cheng, 1981). Because no matrix-digesting enzymes are employed, the basement membrane remains intact, and contamination with stromal cells is thus avoided. This method is very rapid and yields isolation of the entire epithelium of a mouse, for example, within 4 min of initiating EDTA perfusion. Using a highly sensitive RT-PCR method to detect collagen IV as a marker of stromal cells (Weiser et al., 1990; Simon-Assmann et al., 1995), the epithelial preparations are greater than 99% free of underlying stroma.

Further Characterization of Preparations of Intestinal Epithelium

In a specific embodiment of the present invention, there are methods for rapid removal of the entire epithelium (preferably comprising the crypt epithelium) from mouse jejunum. In a specific embodiment, the procedures are preferably compatible with cell viability for tests of stemness. The advantages of the in vivo EDTA method of Bjerknes and Cheng (1981) are discussed elsewhere herein and the present inventor has shown that the epithelium obtained is indeed free of underlying stromal tissue. However, in specific embodiments the epithelial preparations are further characterized to assess both completeness of epithelial recovery and viability.

In specific embodiments of the present invention, the purity of the entire preparation is quantitatively assessed. For example, RNA is prepared from both the released epithelium and the remaining tissue to assess levels of the following exemplary mRNAs: a) sucrase-isomaltase, a marker of villus epithelial cells (Chandrasena et al., 1992; Traber, 1999); b) cryptdin-1, a marker of Paneth cells which reside at the bases of the crypts (Darmoul and Ouellette, 1996; Ouellette and Selsted, 1996); and c) collagen IV, a marker of mesenchymal cells underlying the basement membrane (Weiser et al., 1990; Simon-Assmann et al., 1995). These mRNAs are quantitated, such as by real time quantitative RT-PCR. Preferred separations will comprise undetectable levels of collagen IV mRNA in the epithelial preparation and undetectable levels of sucrase-isomaltase mRNA and cryptdin mRNA in the residual tissue.

Once satisfactory preparations of epithelial cells are obtained, they are used to generate suspensions of single cells. In some aspects of the invention, after brief DNase treatment to prevent clumping (Kaiserlian et al., 1989), these cells are subjected to propidium iodide staining and single wavelength flow cytometry, as described in Example 1, to quantitatively determine the proportion of viable cells.

In the embodiment wherein viability of epithelial cells prepared in this way provides no improvement in viability over the 49% observed in the inventor's original studies using the Evans method, the rapid EDTA method (Bjerknes and Cheng, 1981) is still utilized because it facilitates transplantation on the same day. However, in the embodiment wherein viability from the in vivo EDTA method is much lower than the 49% observed with the Evans method then the Evans method or modifications thereof are utilized. In the embodiments wherein the Evans method is employed, markers of the 10% non-epithelial cells that are expected in the preparation (Evans et al., 1992) are included to identify where they track in the flow cytometry.

### Example 2

### Methods to Generate Single Cell Suspensions of Colon and/or Stomach Tissue Suitable for Flow Cytometry

As with the small intestine, there is a method that yields a viable single cell preparation suitable for flow cytometry from the colon and/or stomach. Two studies were performed with mouse colon using the same digestion method as was used for the jejunum, yet significant clumping was generated, presumably due to mucus from the goblet cells that are more numerous in the epithelium of the colon than of the jejunum. Logical modifications to the method include use of a mucolytic agent such as dithiothreitol and/or N-acetyl-cysteine. In specific embodiments, modifications to the Evans et al. method (1992) are utilized, or a mannitol-based method described by Perret et al. for rat colon (1977) is utilized. Interestingly, this method requires no proteases and yet apparently gives epithelial cells that are totally free from the underlying stroma. The preparations have high viability and good morphology (Perret et al., 1977).

### Example 3

### General Methods for Sorting

Intestinal epithelial cells are suspended at 10⁶ cells per ml in DME containing 2% fetal calf serum (FCS) and 5 μg/ml Hoechst 33342 (Sigma). After incubation at 37° C. for 90 min, they are spun down and resuspended in cold Hank's balanced salt solution containing 2% FCS. Staining with appropriate dilutions of FITC-labeled anti-CD3 and FITC-labeled anti-CD-45 (BD Pharmingen) is performed on ice for 10 min. After washing, cells will be resuspended in Hank's salt solution containing 2 μg/ml propidium iodide to exclude dead cells, and kept at 4° C. until sorting. A 350 nm argon-laser is used to excite Hoechst 33342 and propidium iodide. Analysis is performed on a triple-laser instrument (Cytomation, Fort Collins, USA) at 405/30 and 670/30 nm, as described for bone marrow (Goodell et al., 1996). The side population (SP) is identified and selected by gating on the characteristic emission fluorescence profile of SP cells. A second argon laser, tuned to 488 mm emission, excites FITC. Sorted cell populations are recovered in 100% FCS and kept at 4° C. until processing or grafting.

### Example 4

### Examination of SP Cells for Lineage Markers and Stem Cell Markers

Flow cytometry methods for isolation of viable stem cells from small intestine are described herein. Intestinal epithelial cells are subjected to the same sorting procedures that have been successfully used with bone marrow cells to identify an “SP” population that is highly enriched in putative stem cells. The SP population would then be analyzed for the presence of markers of “stemness” and absence of differentiation markers.

Upon obtaining SP cells, the presence of various lineage markers and/or potential stem cell markers is assayed.

Analysis of Stem Cell Markers and Lineage Markers in SP vs. Non-SP Cells

In the embodiment wherein the SP cells represent intestinal stem cells, they preferably express the stem cell marker Msi-1 (Booth and Potten, 2000). Conversely, the cells are preferentially devoid of markers for each of the principal epithelial lineages. To avoid gut immune cells and circulating bone marrow hematopoietic stem cells confounding the cell separations, these cells are identified by staining with FITC-labeled anti-CD45, and they are eliminated, such as by cell sorting. Use of a triple laser instrument, for example, allows this to occur simultaneously with SP sorting. Although in some embodiments CD45 is a marker for leukocytes, there may be a range of staining intensities. Thus, since IELs constitute the principal immune cells in these epithelial preparations, in some embodiments the cells are also stained with anti-CD3, which is known to identify the vast majority of IELs (Goodman and Lefrancois, 1988; De Geus et al., 1990).

These exemplary methods are designed as follows. Intestinal epithelial cells are collected as described elsewhere herein, then are stained with Hoechst 33342, anti-CD45, and propidium iodide prior to triple wavelength flow cytometry, as described herein. RNA from SP fractions and the entire non-SP fraction is used for quantitative real-time RT-PCR of the following exemplary marker mRNAs: for stem cells, Msi-1 (Booth and Potten, 2000); for the enterocyte lineage, sucrase-isomaltase (Chandrasena et al., 1992; Traber, 1999); for the goblet lineage, intestinal trefoil factor (Mashimo et al., 1995; Matsuoka et al., 1999); for the Paneth cell lineage, cryptdin-1 (Darmoul and Ouellette, 1996; Ouellette and Selsted, 1996). The enteroendocrine lineage is not assessed at this stage, in specific embodiments, because it is a minor component of the total epithelium (approximately 1%) comprised of multiple subtypes (Roth et al., 1990) and thus is unlikely to be informative for the refinement of sorting strategies.

Quantitative RT-PCR is preferable for these analyses for two reasons. First, the high sensitivity ensures ease of detection of the various mRNAs within even the smallest fraction of cells (lower SP). Second, the quantitative nature of the data allows calculation of the proportion of each transcript found in each of the cell populations (SP and non-SP). This, in turn, is valuable in assessing the nature and purity of each fraction.

Given that an additional/alternative stem cell marker would be of great value, in specific embodiments of the present invention both in situ hybridization and immunohistochemistry, for example, are utilized to determine the pattern of expression of Brcp1 in the mouse intestine. Brcp1 is the ABC transporter that has recently been found to account for the SP phenotype in both bone marrow and skeletal muscle (Zhou et al., 2001). Brcp1 protein and/or RNA may be localized to the stem cell zone and thus would constitute a valuable exemplary marker. For example, antibodies to mouse Brcp1 may be used for immunohistochemistry of Brcp1 protein, and/or RT-PCR may be used to show that Brcp1 is expressed in the small intestine, such as by in situ hybridization.

### Example 5

### Isolation of Intestinal Epithelial Cells, SP Sorting, and Analysis of Marker Gene Expression

The disclosure of the invention describes isolation of intestinal stem cells, such as in part by SP sorting, followed by analysis of particular markers, such as by analysis of their expression, including at least analysis for their presence and/or level, in specific embodiments.

Generation of Viable Preparations of Isolated Epithelial Cells

Mucus-free single cell suspensions of mouse intestinal epithelium were produced, and the cells remained viable through the manipulations required for preparation, staining and sorting. To this end, two dissociation methods were studied: the dispase/collagenase method of Evans et al. (1992) and the in vivo EDTA method of Bjerknes and Cheng (1981). Although the EDTA method in some embodiments is favorable, based on its rapidity and on the lack of stromal contamination, reproducible results were difficult to obtain with this method. Most notably, although villus epithelial cells were always readily removed, the efficiency of removal of the crypt epithelial cells was highly variable. In addition, viability of the cells prepared with EDTA was less than desirable. However, in specific embodiments this method is utilized to isolate IESCs for the present invention.

Thus, the present inventors focused their efforts on streamlining and perfecting the Evans' method. Modifications to this method permit reliable mucus-free single-cell suspensions of high viability (87% before staining and 61% after staining), as opposed to the production of organoids by the Evans method. For comparison, with the EDTA method viabilities averaged 70% before staining and fell to 36% after staining.

Side Population Sorting

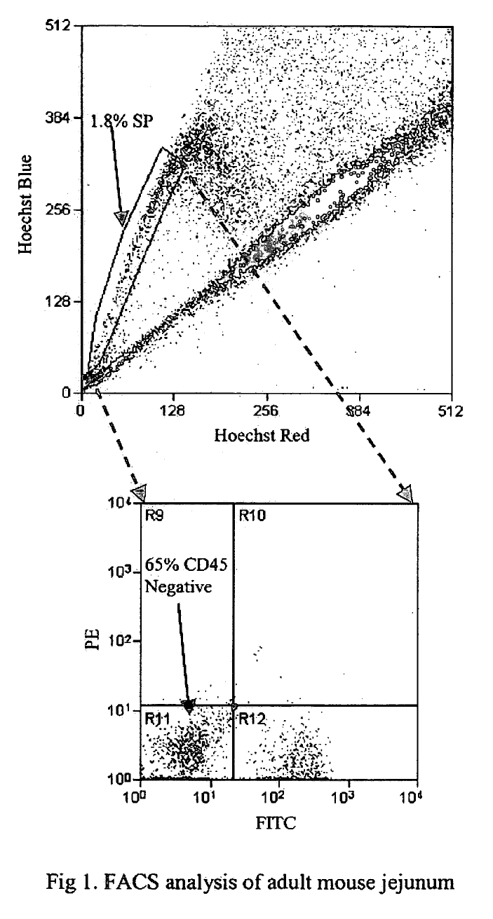

As described elsewhere herein, the cell suspensions were stained with Hoechst 33342 and co-stained with both FITC-labeled anti-CD45 to eliminate cells of hematopoietic origin and with propidium iodide (PI) to identify dead cells. Typical results of dual wavelength flow cytometry of the viable cells based on the Hoechst fluorescence are shown in FIG. 1. As can be seen (top panel), a distinct side population (SP) was identified. In the preparation shown, the SP comprised 1.8% of the viable cells. Further analysis of the SP is shown in the lower panel, where it can be seen that the SP includes two distinct populations of CD45-positive and CD45-negative cells. The latter, which comprised 65% of the SP in the case shown, are herein classified as IESCs. In contrast, the CD45-positive SP fraction likely comprises either intraepithelial lymphocytes or hematopoietic stem cells that are known to circulate in substantial numbers (Wright et al., 2001).

Since perfecting the cell isolation step, at least 56 SP analyses with adult mouse jejunum have been performed. The composite results from all of these preparations are shown in Table 1.

TABLE 1
Composite Data from Preparations of Adult Mouse Jejunum

| | Collagenase/Dispase | EDTA/Dispase |
|---|---|---|
| | n = 40 | n = 11 |
| Viability Before Hoechst (%) | 85.0 ± 0.8 | 78 ± 2.4 |
| Viability After Hoechst (%) | 66.0 ± 0.7 | 59 ± 2.4 |
| SP as fraction of viable cells (%) | 3.3 ± 0.3 | 3.0 ± 0.7 |
| CD$%-negative component of | 35.0 ± 2.5 | 60 ± 7.4 |
| SP (%) | | |
| CD45-negative SP as frac- | 1.1 ± 0.1 | 2.1 ± 0.6 |
| tion of viable cells (%) | | |

It is noteworthy that although viability falls somewhat after the Hoechst staining, as has been reported with other tissues, it still remains impressively high. Furthermore, the abundance of CD45-negative SP cells is remarkably close to the upper limit of the range predicted for intestinal stem cells (0.8%). In terms of absolute numbers, at least 10,000 viable CD45-negative SP cells per mouse jejunum were obtained.

In an embodiment wherein IESC from suckling mice have greater regenerative potential than those from adult mice (Tait et al., 1994; Kaihara et al., 2000; Tavakkolizadeh et al., 2003), SP sorting is applied to the suckling intestine. To this end, the same digestion was performed, followed by staining and flow cytometry methods with jejunum from mice at postnatal days 7, 8 and 12. In all cases, a viable CD45-negative SP has been observed, with the numbers of cells in this fraction being within the same range as those reported in Table 1 for adult jejunum. Interestingly, viability after Hoechst staining was 72-75%, which is somewhat higher than the average seen with adult preparations.

Analysis of Marker Gene Expression in SP as Compared with Non-SP Cells

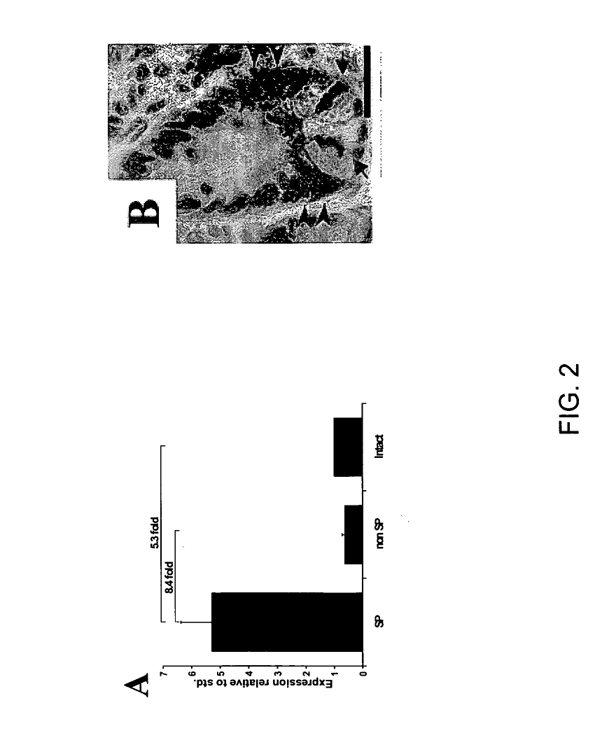

Msi-1 is a preferable marker of IESCs. To assess the expression of Msi-1 in the cell fractions described herein, following flow cytometry RNA was prepared from CD45-negative SP and non-SP fractions using RNAqueous-Micro Kit (Ambion, Inc., Austin, Tex.) according to manufacturer's directions. The concentration of isolated total RNA was calculated using a NanoDrop 1000 (NanoDrop Technologies, Inc., Rockland, Del.). Quantitative real time RT-PCR (qRT-PCR) was performed with 1 ng of RNA using the Taqman One-step RT-PCR Master Mix Reagents Kit (Applied Biosystems) on an ABI Prism 7700 instrument under standard conditions (PE Applied Biosystems, 2001). Data were analyzed using the ΔΔCt method (PE Applied Biosystems, 2001) with 18S rRNA as the constitutive marker and using RNA from intact mouse jejunum as the reference. The results are shown in FIG. 2A. As can be seen, Msi-1 mRNA is greatly enriched in the SP fraction: 5-fold over the concentration seen in intact tissue and 8-fold over that in non-SP cells. For reference, the immunohistochemistry data of Kayahara et al. (2003) are shown in FIG. 2B. The marked enrichment of Msi-1 in the SP fraction is considered strong evidence that this fraction includes the IESCs. In specific embodiments of the invention, the strong enrichment of Msi-1 in the CD45-negative SP fraction indicates that the SP fraction comprises the majority of stem cells.

Since the Evans' dissociation method is known to give epithelial cells contaminated with approximately 10% underlying stromal cells (Evans et al., 1992), and since Msi-1 has been detected in a few cells within the lamina propria (Potten et al., 2003; Kayahara et al., 2003) the present inventor verified that the SP cells were not stromal cells. To this end, RNA from SP cells was analyzed for collagen IV mRNA, a known marker of intestinal stromal cells (Simon-Assmann et al., 1995; Weiser et al., 1990) including the pericryptal myofibroblasts (Pourreyron et al., 2003; Evans et al., 2003). No collagen IV signal was detected by qRT-PCR even after 40 cycles. Thus, the Msi-1 positive SP cells are indeed epithelial and comprise the IESCs.

Similar qRT-PCR analyses are utilized for a variety of other mRNAs. Specifically, Hes-1 and EphB2, which are both reported to be abundant in the stem cell zone but are also expressed in a decreasing gradient in the cells immediately above that zone (Batlle et al., 2002; Kayahara et al., 2003). Analysis of these mRNAs will allow some assessment of the extent to which the SP includes the transit amplifying cells, as well as the IESCs.

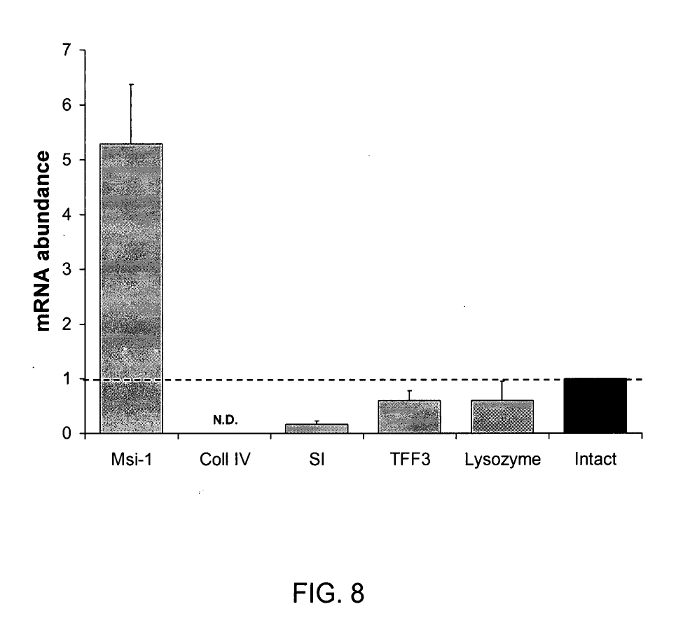

In other embodiments, the assessment of the extent of contamination of the SP by underlying stromal cells and by differentiated epithelial cells we subjected SP mRNA to qRT-PCR for collagen IV (stromal marker), sucrase-isomaltase (enterocyte lineage), intestinal trefoil factor-3 (goblet cell lineage) and lysozyme (Paneth cells). The data are shown in FIG. 8, with the Msi-1 data from FIG. 2 replicated for comparison. As can be seen, collagen IV was undetectable (even after 40 cycles) and all three lineage markers were de-enriched in the SP as compared with intact jejunum. In addition, the expression of markers is examined for each of the four epithelial lineages that should be de-enriched in the SP fraction.

Thus, in additional embodiments the SP, although clearly enriched in IESCs as evidenced by the Msi-1 data, may comprise traces of each of the differentiated lineages. In embodiments the SP cells are further purified, such as, for example, by using antibodies.

### Example 6

### Development of an Animal Model with Local, Limited Sterilization of Intestinal Crypts

As described in this Example, there is development of an animal model with local, limited sterilization of intestinal crypts, in particular embodiments to generate an ideal denuded region for subsequent transplantation of putative stem cells. In particular embodiments of the invention, the parameters used for the model are extrapolated and optimized for use in a human.

As discussed elsewhere herein, in some aspects intestinal epithelial stem cells will not differentiate in vitro. Provided that appropriate mesenchymal support is available, differentiation into all four epithelial lineages has been observed in vivo (specifically, after grafting under the kidney capsule, subcutaneously or into mucosectomized colon). In specific embodiments, the small intestine itself is utilized as a favorable site to characterize survival and differentiation of putative stem cells. To facilitate generation of a system for transplantation, cytotoxic drugs that are known to be capable of destroying both stem cells and clonogenic cells are exposed to the small intestinal crypts. Such drugs selectively cause apoptosis of the proliferating epithelial cells without affecting the matrix and underlying mesenchyme (Ijiri and Potten, 1983; Anilkumar et al., 1992; Okudela et al., 1999; Al Dewachi et al., 1980; Der et al., 1975; Wright et al., 1989). Moreover, migration of healthy cells from the upper crypts toward the villi continues despite the presence of the cytotoxic drugs (Ijiri and Potten, 1983), thus exposing the basement membrane in the proliferative zone. In preferred embodiments, this favors both engraftment and differentiation following transplantation of stem cells. Since in one aspect of the invention the stem cells are drawn to the crypt base by chemotaxis, a further advantage of a damage model that leaves the matrix intact is that chemotactic migration of intestinal epithelial cells has recently been shown to be matrix dependent (Polk and Tong, 1999).

Choice of Cytotoxic Agent

In the 1980s, several laboratories investigated the effects of more than 18 cytotoxic drugs on the crypts of the mouse small intestine following i.p. administration (Al Dewachi et al., 1980; Ijiri and Potten, 1987; Moore, 1985). Aspects of the damage process that were carefully documented include: a) the spatial distribution of cell death within the crypt; b) the time course of cell death; and c) the ability of crypts to recover from the insult. Broadly speaking, these studies showed that cytotoxic drugs fall into at least two categories. The first group of drugs have their most dramatic effect in the mid-crypt region and thus cause depletion of the clonogenic cells but apparently spare at least one stem cell per crypt, as crypts are typically able to rejuvenate. This group of drugs includes vincristine, cyclophosphamide, arabinoside-C, and hydroxyurea (Al Dewachi et al., 1980; Ijiri and Potten, 1987; Moore, 1985), for example. The second group of drugs (which includes isopropyl-methane-sulphonate, bleomycin and adriamycin, for example) cause death of both stem cells and clonogenic cells and thus lead to complete sterilization of crypts (Ijiri and Potten, 1987; Moore, 1985). Admittedly these groupings are not totally exclusive because there are some drugs (e.g. 5-fluorouracil and methotrexate, for example) that cause maximum cell death in the mid-crypt (Ijiri and Potten, 1987) and yet are capable of crypt sterilization (Moore, 1985). However, dose response studies (Moore, 1985) show that crypt sterilization by the latter drugs requires relatively high doses. In contrast, drugs from the second category are capable of sterilizing a significant fraction of the total crypts at relatively low doses (Moore, 1985). Thus, drugs from this category are viewed as ideal agents to cause local destruction of both stem cells and clonogenic cells prior to transplantation of preparations of putative stem cells. As noted above, although these drugs denude crypts, they do not disrupt the basement membrane or the underlying mesenchymal cells (Ijiri and Potten, 1983; Anilkumar et al., 1992; Okudela et al., 1999; Al Dewachi et al., 1980; Der et al., 1975; Wright et al., 1989). Thus, introduced stem cells, in preferred embodiments, have maximal opportunity to seed into the crypt base, to proliferate and to generate progeny capable of differentiation.

Amongst the group of cytotoxic drugs known to have maximal effects in the lower crypt (Ijiri and Potten, 1987) and to be capable of crypt sterilization (Moore, 1985), the present inventor has chosen to employ adriamycin and bleomycin, for purposes of example. Compared with other drugs in this group, the further advantages of these two include: a) both show very rapid action following i.p. administration, leading to complete cessation of mitosis within 3 hr (Ijiri and Potten, 1987); and b) dose-response studies (Moore, 1985) have shown that both adriamycin and bleomycin are less deleterious to bone marrow than to crypt cells following systemic administration. The former feature (rapid action) means that there is high likelihood that they are effective within the proposed 4-hr clamped period. The second feature means that even if appreciable amounts are absorbed from the clamped segment, at the doses proposed there should be minimal effects on bone marrow. This is in contrast to other cytotoxic drugs (e.g., 5-fluorouracil) that would fulfill the criterion of crypt sterilization and rapid action but that are very much more toxic to bone marrow than to the intestine (Moore, 1985).

Both adriamycin and bleomycin are employed, because each shows potential advantages. Adriamycin is extensively removed by the liver (60% in a single pass). Thus, it could be administered in high doses within the clamped segment and yet, even if absorbed, would give relatively low levels in the systemic circulation (because of delivery to the liver via the portal vein), therefore producing minimal effects on bone marrow and the rest of the GI tract. Bleomycin has minimal toxicity to bone marrow even when administered systemically (Goodman and Gilman, 1975). As a consequence, the i.p. LD₅₀ for mice (210 mg/kg) is almost 4-fold higher than the i.p. dose known to cause sterilization of 50% of intestinal crypts (see below).

Dose Response to Adriamycin and Bleomycin in Clamped Segments

Following i.p. administration to adult mice, there is a log linear relationship between crypt survival and drug dose (Moore, 1985). The present inventor envisions that a similar dose-response relationship can be established following administration into clamped segments of the small intestine. Thus, various doses of each drug are used in order to establish the dose-response relationship and to identify a dose that causes sterilization of approximately 50% of the crypts in that segment. The latter has been chosen as a target because (in the absence of transplanted cells) the literature indicates the gut can recover from this extent of damage (Wright, 2000; Moore, 1985). Moreover, when utilized in transplantation methods, the 50% crypt-sterilized segment should provide ample sites for engraftment of introduced stem cells and an ideal rejuvenative environment (Wright, 2000; Houchen et al., 1999).

In a specific embodiment, the i.p. doses are established using appropriate methods. From the dose-response data of Moore (1985), the i.p. doses of adriamycin and bleomycin that cause sterilization of 50% of crypts are 15 mg/kg and 60 mg/kg, respectively. In particular aspects of the invention, 100%, 30% and 10% of these doses are delivered to a clamped segment of the jejunum. Both drugs are soluble in normal saline (Moore, 1985), which may be used as a pharmaceutically acceptable excipient.

Detailed procedures for these clamped segments are given elsewhere (Lau et al., 1995). Briefly, a laparotomy is performed under isofluorane anesthesia. A 2-cm piece of the jejunum is cannulated and flushed with warm isotonic saline, palpated to ensure removal of saline, and then clamped for drug delivery via a 27-gauge needle. To prevent thermal shock, drug-filled syringes are kept at 37° prior to use. The laparotomy and skin incision are sutured closed, and the animal is allowed to regain consciousness for the duration of the 4-hr period. At the end of this time period, there is another brief isofluorane anesthesia, during which the original incisions are opened and the clamps are removed. In addition, at this time the location of the clamps is indicated by a small piece of 5-0 silk.

In order to quantitate crypt sterilization, assessment is made at a time when non-sterilized crypts have had adequate opportunity to rejuvenate. As the peak of mitotic recovery following i.p. injection of these drugs occurs after 4 days (Moore, 1985), in specific embodiments this is also an appropriate time to examine the tissue after intraluminal administration. At the time of sacrifice, trunk blood is collected, and the segment is clamped, as is an adjacent upstream control segment. Analogous tissues are collected from sham-operated animals that receive normal saline rather than drug in the clamped segment.

Each mouse receives BrdU (120 mg/kg) and 5-fluoro-2′-deoxuridine (12 mg/kg) 2 hr before death to label S phase cells. Both the clamped segment of intestine and the control segment are processed for routine histology and BrdU immunohistochemistry. Crypt survival is assessed by the microcolony survival assay (Withers and Elkind, 1970) as modified by Houchen et al. (Houchen et al., 1999). In this assay, the number of regenerating crypts in each transverse section is compared with that in control animals. Data from the upstream segment of each experimental animal provides a measure of the extent of drug effects in the remainder of the GI tract as a result of systemic delivery following absorption. In addition, hematocrits are performed on blood samples in order to monitor deleterious effects on bone marrow.

In particular embodiments, the doses are optimized such that they are in the range to cause sterilization of 50% of crypts. In the embodiment wherein neither drug permeates the intestine from the luminal side, for example, systemic administration is employed, and bleomycin alone may be particularly utilized because of its minimal toxicity to bone marrow (Goodman and Gilman, 1975). Also, in this embodiment there may be only 20-30% crypt sterilization, such as to minimally compromise the remainder of the GI tract but still offer adequate engraftment opportunities for stem cell preparations.

Time Course of Response to Intraluminal Adriamycin and Bleomycin

In a specific aspect of the invention, 4 days is the time needed to maximize the opportunity for crypt regeneration and thus to assess crypt sterilization accurately. However, for the purposes of transplantation and, in some embodiments, the earliest time at which crypt cell death has plateaued is determined, which may be optimal for stem cell engraftment. On the other hand, to the extent that damaged crypts lead to the elaboration of critical growth and survival factors (Wright, 2000; Houchen et al., 1999), an alternative ideal time for transplantation would be when the nonsterilized crypts are showing a maximal proliferative response. Although the time courses of both cell death and mitotic recovery have previously been established for i.p. administration of adriamycin and bleomycin (Ijiri and Potten, 1987), for example, these time courses may need to be optimized following intraluminal administration, and optimization is well within the skill level of one in the art.

The studies may be designed as such. The experiment described above is repeated using the single doses of adriamycin and bleomycin that are predicted to cause sterilization of approximately 50% of crypts. Because crypt proliferation undergoes quite marked circadian variation (Al Nafussi and Wright, 1982; Potten et al., 1977), surgery is performed on experimental and sham-operated animals in a pair-wise fashion. Animals are sacrificed 1, 2, 3 and 4 days after drug administration (or sham-operation) using the BrdU protocol described above. Following routine histology and immunohistochemistry, the numbers of dying cells and proliferating cells per crypt are scored as described by Ijiri and Potten (1987). Briefly, using cross-sections of intestine and selecting crypts showing good longitudinal sections (i.e., evidence of a lumen, some Paneth cells at the base, and at least 17 cells along the side), the numbers of dying cells and proliferating cells are scored in 50 half-crypt sections of each mouse. In addition, in view of the importance of matrix/mesenchyme as environment for transplanted stem cells, the intactness of the basement membrane is also assessed in the same sections. Scoring is performed independently by two observers, both of whom are blinded to the identification of the tissue.

In a preferred embodiment, drug-induced cell death and the mitotic response of surviving crypts plateau at the same time, and this time point is preferable for stem cell transplantation. In the embodiment wherein these events are discoordinate, more than one time point may be utilized in the transplantation studies.

### Example 7

### Additional Animal Model with Double-Balloon Catheter

An additional model is provided herein. In particular embodiments of the invention, the parameters used for the model are extrapolated and optimized for use in a human.

As indicated earlier, clamped segments are one preferred surgical approach primarily because their simplicity allows reasonable numbers of animals to be processed and studied. Although the inventor's experience to date with clamped segments had minimal morbidity or mortality, in some embodiments even the modest damage desired renders the gut sufficiently fragile as to react badly to a subsequent surgery (i.e. for delivery of stem cell preparations, for example). For this embodiment, an alternate surgical approach that obviates gut manipulation at the time of cell delivery is employed.

In particular embodiments, a double-balloon catheter that has been devised by Dr. Michael Helmrath is employed. The catheter comprises balloons that when inflated will delineate a section of small intestine (for example, a section no less than about 1 cm and no greater than about 10 cm in a mouse, such as about 2 cm), a delivery tube with multiple outlets within the ballooned section and a port which will be exteriorized at the back of the neck. An analogous length is delineated in a human small intestine section. The entire device is sufficiently small as to not obstruct intestinal flow when the balloons are deflated. In a specific aspect of the invention the dose and time studies described above are repeated therewith An advantage of the catheter over clamped segments is that, if necessary, the dose could be administered in multiple installments, and this may be preferable to achieve the desired level of intestinal damage with minimal systemic effects. Likewise, when the catheter is used to deliver stem cell preparations to the damaged region, it offers greater flexibility of the duration of the seeding period as well as the possibility of repeated seeding. Since the catheter is removed after delivery of stem cells, the fate of these cells can be studied for the life of the animal without further manipulation.

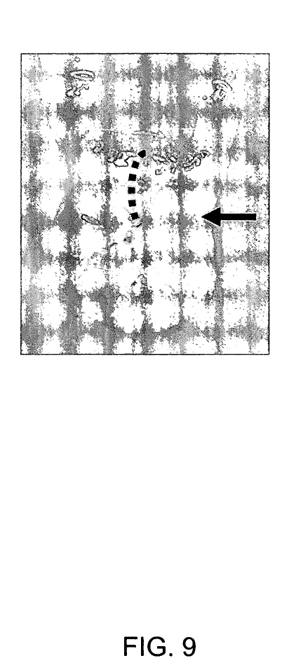

Another embodiment uses a temporary intestinal bypass, also devised by Dr. Michael Helmrath. The procedure begins with DOX delivery to a clamped segment (5-7 cm) of small intestine as described in Example 6 above, for example. At the end of the 4 hr damage period the treated segment is converted into an in continuo loop via anastomosis of the gut wall at the proximal and distal ends of the segment (see FIG. 9). To remove all traces of DOX, the segment is flushed three times, first with warm PBS, then with a mucolytic solution (Eckert et al., 1995), then with PBS again. A piece of rice is inserted at the distal end of the loop, causing a temporary obstruction of the bypassed loop, while the side-to-side anastamosis allows continued transit of intestinal contents. Stem preparations can then be delivered to the loop and will have a prolonged period of time in which to engraft. Preliminary experiments suggest that the rice swells to maintain a temporary blockage and then dissolves by 72 hrs. Very little diet is seen within the loop at this time, indicating that most of the enteral content is still going through the side to side anastomosis. By 7 days the loop is full of diet, indicating that the normal aboral route is re-established by this time. To date Dr. Helmrath has performed this surgery on 20 mice as sham operations (i.e. no SP cells) designed to optimize the temporary bypass. Animals were killed at 3, 7, 28 and 42 days after surgery and only 1 of the 20 mice died before their planned date of sacrifice. The condition of the bowel loop was excellent at all times (FIG. 9, for example, was taken 28 days after surgery and is typical).

Engraftment of ROSA 26 Cell Preparations in a Damaged Segment of Jejenum

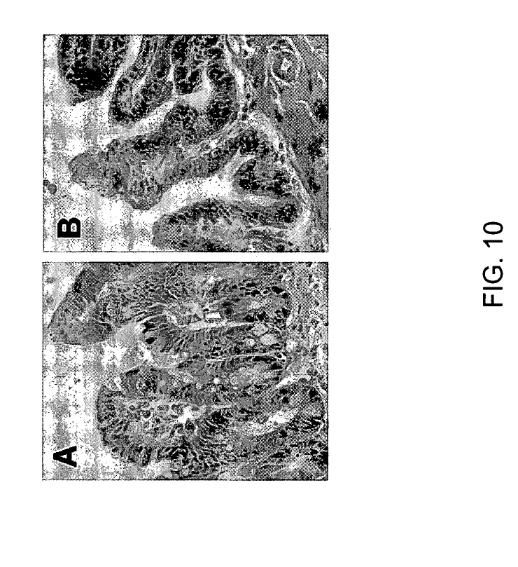

In order to verify that the ROSA 26 strain on the C57B1/6J background is compatible with wild type C57B1/6J mice for purposes of intestinal cell transplantation, the present inventor utilized a mice model in which a segment of intestine was fully bypassed. In this embodiment, the dispase/collagenase digests were the starting material for cell sorting, although in alternative embodiments the SP cells are the starting material. Thus, bypassed jejunal segments were created in wild type mice and cell digests prepared from ROSA 26 jejunum were delivered luminally. Experimental segments were harvested at various times after transplantation and subjected to X-gal staining. There was evidence of engraftment at all times. Results from the 21 day time point are shown in FIG. 10. As can be seen in panel A of FIG. 10, there were patches of X-gal positive epithelium with well formed villi and crypts. Other areas (panel B of FIG. 10) showed only wild-type epithelium.

Although in some aspects of the invention these engraftment methods are employed, in alternative embodiments other engraftment procedures are utilized, such as those described above and illustrated in FIG. 9, for example.

### Example 8

### Transplantation of Intestinal Stem Cells into Denuded Regions of Small Intestine and Assessment Thereof

Transplantation of intestinal stem cells into denuded regions of small intestine and subsequent assessment of the morphology as well as the expression of lineage-specific markers in epithelium derived from the transplanted cells is described herein this Example.

As described elsewhere herein, the assessment of sternness in cell populations is preferably performed in vivo. In specific embodiments, small intestinal segments with approximately 50% crypt sterilization provide the optimal environment for seeding, proliferation and differentiation of transplanted intestinal stem cells. For the purposes of transplantation, the critical elements of the damage model described above include at least: a) the presence of normal matrix and mesenchymal components in the denuded crypts; and/or b) the lack of endogenous stem cells and clonogenic cells that would otherwise compete with the transplanted cells.

Identification of Transplanted Cells

In order to track the fate of transplanted cells and their progeny, it is essential to have some method to distinguish donor from host cells. In the studies described herein, in addition to characterizing differentiation of progeny of putative stem cells into all four epithelial lineages, it is also valuable to make quantitative assessments of the extent of contribution of donor cells to the overall regeneration of the epithelium. This is preferentially achieved by whole mount staining that allows whole fields of villi and underlying crypts to be observed. As noted previously, differential surface staining by lectins were essential to the use of mouse chimeras in the study of crypt clonality and in mutagenesis studies of crypt cell lineages (Gordon et al., 1992). However, these lectin-binding strains of mice are not optimal donor animals in the present invention, because the host strain (no lectin binding) would not be syngeneic and thus would risk a high rate of graft rejection.

A more favorable alternative is the use of ROSA 26 mice for the preparation of putative intestinal stem cells. These mice have widespread expression of the bacterial β-galactosidase (β-gal) gene (Zambrowicz et al., 1997), including all epithelial cells of the small intestinal epithelium (Wong et al., 1996; Wice and Gordon, 1998). Moreover, studies with ROSA 26 chimeric mice have shown: a) successful staining of whole mounts; b) absence of background staining on non-ROSA cells; and c) easy distinction of ribbons of β-gal positive cells emanating from ROSA crypts (Wong et al., 1996; Wice and Gordon, 1998). ROSA 26 mice on a C57B1/6J background, for example, may be employed, given that they have been routinely used for successful transplantation of stem cells from various tissues into C57B1/6J hosts (Wulf et al., 2001; Jackson et al., 2001).

Although appropriate positive (ROSA) and negative (C57) control tissue is run with each batch of β-gal staining, in the event that any background staining becomes an issue, it is advantageous to be able to independently identify the transplanted cells. To this end, donors are preferentially male mice and recipients are preferentially female so that donor derived cells can be identified in tissue sections by in situ hybridization with a probe recognizing repetitive sequences on the Y chromosome (Eckert et al., 1995), which has been used in prior transplantation studies.

Assessment of Engraftment and Morphology After Transplantation of Putative Intestinal Stem Cells

Although in particular aspects of the invention the detailed lineage analysis is preferential in order to identify stemness, in some aspects the analysis is not completed in full or at all, but a successful engraftment is generated.

One exemplary design of the studies is as follows. Host female C57B1/6J mice are pretreated by local or systemic drug administration using dose and time established as described above. The appropriate population of cells that are good candidates for intestinal stem cells, such as the SP cells (or some portion thereof), is prepared and collected from the jejunum of male ROSA 26/C57B1/6J mice. For comparative purposes, the non-SP fractions of cells are also collected. Groups of 3 mice receive either: a) no cells (PBS vehicle only); b) LSP cells at the same or similar dose; or c) non-SP cells at about a 10-fold higher dose, i.e. about 80,000 cells. Cells are delivered to clamped segments as described for drugs above. If the pretreatment is via the local route (rather than systemic), to ensure that all cells are within the damaged region, the second set of clamps are placed approximately 0.5 cm inside the threads marking the original placement of clamps. This provides a 1 cm segment for transplantation. The volume delivered to the segment is adjusted to give modest distension in order to improve access to the denuded crypts (Sandberg et al., 1994). Initial studies having no damage indicated this volume to be 0.05 ml, but it may be greater following drug treatment, in specific embodiments of the present invention.

Clamps are removed after 4 hr. Based on impressive rates of chemotactic migration reported for intestinal epithelial cells (Polk and Tong, 1999), in some embodiments the stem cells find their way to the bases of the denuded crypts within the 4-hr period.

Animals are sacrificed 7 days after transplantation, and the entire 1-cm transplanted segment is subjected to whole-mount staining for β-gal, as described in studies from the Gordon lab (Wong et al., 1996; Wice and Gordon, 1998). Successfully engrafted stem cells are detectable, such as by ribbons of blue-stained cells emerging onto unstained (host) villi. The numbers of such positive ribbons are counted and compared with an estimate of the total number of crypts within each field. After whole-mount examination, the tissue is paraffin embedded, sectioned and stained with nuclear fast red as described by Wice and Gordon (1998) to assess cellular morphology and crypt/villus architecture. In embodiments wherein the ability of the X-gal stain to clearly distinguish donor from host cells is uncertain, serial sections are used for in situ hybridization to detect the Y chromosome.

Based on published estimates of the numbers of crypts per cm² in the mouse jejunum (Cheng and Bjerknes, 1985; Hagemann et al., 1970), it is predicted that the number of crypts in a 1-cm segment is approximately 16,000. If 50% of these are sterilized (as planned for the model), there would be about 8,000 crypts available for engraftment. Thus, as a starting dose, this number is envisioned to deliver of the putative stem cells (SP fraction). Even in cases where few of the denuded crypts show engraftment, i.e., 0.5% of total crypts there is no difficulty detecting these (i.e. 80 ribbons per segment), as whole mount staining is in fact capable of detecting a single ribbon within the entire intestine (Gordon et al., 1992).

In the embodiment wherein no β-gal-positive cells are observed, the study is repeated using 10-fold higher starting numbers of SP cells. Depending on yields, this may require up to 2 mouse donors per recipient. If reasonable numbers of β-gal-positive cells are observed and if histology indicates that normal morphology has been achieved at this time (7 days post-transplantation), further lineage analysis will occur. In the embodiment wherein there are reasonable numbers of positive cells but abnormal morphology (e.g., patches that have not yet fully emerged from the crypts), the study is repeated with harvest at later times (e.g., 14 days).

In the embodiment wherein the non-SP fraction excludes both stem cells and clonogenic cells, there may be no engraftment. Here again, particular timing of the experiment may be required. At very early times (e.g. about 1 day) there might be some non-specific adhesion to the denuded basement membrane, but in about 7 days true engraftment would not be expected.

In the embodiment wherein systemic administration of bleomycin as employed as the preferred route of damage prior to grafting, focus on sterilization of only 20-30% of the crypt is undertaken in order to avoid compromising the entire GI tract. As can be seen from the calculations above, even at 20% there would be approximately 3200 crypts available for engraftment per 1 cm segment and if, in the worst case, only 1% of these show engraftment, there would still be approximately 30 ribbons of blue cells per segment. In fact, in view of these favorable calculations; even for local drug delivery the present inventor envisions grafting outcomes with 20-30% crypt sterilization, instead of 50%.

Further Analyses of Lineages Arising from Transplanted Intestinal Stem Cells

The routine histology performed as described above gives a good indication of whether or not the transplanted cells are capable of differentiation into various lineages (based on distinct morphology of enterocytes, goblet cells and Paneth cells following nuclear fast red staining, for example). Thus, once optimal transplantation conditions are established (number of cells, timing, etc.), more in-depth analysis of the epithelial lineages are utilized.

One exemplary design of these particular studies is as follows. Tissue sections already stained with X-Gal and judged to be optimal from studies described above are subjected to further histochemistry as follows: a) periodic acid/Schiff (PAS), which detects neutral mucins present in goblet cell mucus globules and Paneth cell granules, as well as glycoconjugates associated with the apical brush border membrane of enterocytes; b) phloxine/tartrazine, which stains the apical secretory granules of Paneth cells; and c) the Grimelius silver stain, which detects enteroendocrine cells. These stains have been used for lineage detection in previous studies from the laboratory of the present inventor (Oesterreicher et al., 1998). More importantly, Wice and Gordon (1998) have reported that when used on ROSA 26 chimeric mice, the intracellular X-Gal precipitates are not removed by the subsequent staining procedures, thus allowing detection of specific cells within β-gal-positive ribbons.

In embodiments wherein histochemistry demonstrates a desired outcome, further analyses using immunohistochemistry on the same X-Gal-stained sections are employed, such as described by Wice and Gordon (1998). Based on lineage analyses by these (Wice and Gordon, 1998) and other (Quaroni and Hochman, 1996; Verburg et al., 2000) authors, immunostaining is employed with the following antibodies: a) for enterocytes, anti-sucrase-isomaltase and anti-apolipoprotein B; b) for goblet cells, anti-mucin-2 and anti-mouse intestinal trefoil factor; c) for Paneth cells, anti-cryptdin and anti-lysozyme; and/or d) for enteroendocrine cells, anti-serotonin, anti-CCK and anti-GIP. The latter choice is based on the observation that, of the 15 more different types of enteroendocrine cells present in the gastrointestinal tract (Dobbins et al., 1991), the most abundant in the mouse jejunum include serotonin, CCK and GIP secreting cells (Roth et al., 1990).

In addition to the lineage assessments, in order to reveal patterns of proliferation within crypts that have regenerated from transplanted cells, the cells are stained with antibodies to Ki67. The present inventor (Oesterreicher et al., 1998) and others (Wright, 2000; Clatworthy and Subramanian, 2001; Korinek et al., 1998) have previously shown that these antibodies readily detect proliferating cells in mouse intestinal crypts. =p In the embodiment wherein acceptable morphology is observed, in a specific embodiment most or all lineages are successfully documented by methods described above or analogous thereto. In the embodiment wherein there is engraftment but no evidence of differentiation into any of the four lineages, it is prudent to include a control sample of animals transplanted with non-SP cells in the event that engraftment is nonspecific (i.e., not necessarily a property of stem cells). In one specific embodiment, there is differentiation into just one lineage, such as, for example, either enterocytes or goblet cells, because Bjerknes and Cheng (1999) have shown that mouse crypts have, in addition to multipotent stem cells, relatively long-lived unipotent progenitors of both of these lineages. In this embodiment, further analysis of the expression of specific lineage markers in the LSP cells prior to transplantation is performed.

Long-Term Survival of Transplanted Stem Cells

In one aspect of the invention, the longevity of the transplanted cells is characterized. There is some debate as to whether or not endogenous stem cells survive for the entire life of the mouse (Winton, 2001). Recent mutagenesis studies (Bjerknes and Cheng, 1999) show clearly that they last at least 154 days (i.e., approximately 5 months). In terms of therapeutic application, these long-term studies are beneficial for assessing excessive proliferation by transplanted cells. For example, this would be apparent if crypts arising from transplanted cells were either excessively large or excessively numerous (e.g., as a result of crypt fission), which is well known to occur in response to increased cellularity (Wright, 2000). Excessive proliferation is also indicated by the presence of proliferative cells in the upper third of the crypts and on the villi.

One exemplary design for these studies is as follows. Using optimal conditions devised above, about three or more groups of animals are studied with the intent to sacrifice at 1, 2, and 5 months post-transplantation. Engraftment, proliferation and differentiation will be assessed as described elsewhere herein.

The long-term outcome depends to a large extent on the success of the damage model. In the embodiment wherein a proportion of crypts are truly sterilized, stem cells that engraft in these crypts should have a good chance of long-term survival. In an alternative embodiment wherein the crypts have surviving endogenous stem cells or clonogenic cells, then it is likely that the transplanted cells are at a competitive disadvantage. In this embodiment, there may be good initial engraftment but subsequent loss, analogous to the process of stem cell “purification” that occurs during the first two postnatal weeks (Gordon et al., 1992). The result of such a process would be that at later times there would be intestines with normal numbers of crypts but diminished marker (such as X-Gal) staining, whereas with losses due to reduced longevity of transplanted cells, there would be a reduction in the total number of crypts per cross-section (analogous to the situation immediately after drug administration).

In other aspects of the invention, there is one or more drastic damage models in which the underlying basement membranes and mesenchymal cells are disrupted. One important aspect for therapeutic application is whether transplanted stem cells are able to gain support from mesenchymal cells anywhere within the lamina propria, as opposed to those specific cells underlying the basement membrane of the crypts. Likewise, the invention encompasses heterologous transplantations (e.g., jejunal stem cells into either ileum or colon) in both minimal and maximal cell damage models. For particular aspects of the invention, in a minimal damage model the mesenchymal environment dictates the differentiated phenotype of the epithelium. With greater damage, the instructive effect of mesenchyme may be lost, but so may be the entire capacity for differentiation.

Alternate Approach to Study “Stemness”

In the embodiment wherein the SP fraction includes most of the stem cells (as determined by presence of Msi-1 mRNA, for example), but there is no engraftment by this fraction, this crypt damage model may be considered non-optimal for demonstrating “stemness”. In this embodiment, ectopic transplantation (e.g. subcutaneous) is employed. To date, embryonic intestinal mesenchyme is the only source of mesenchyme that has been shown capable of supporting differentiation of isolated epithelial cells into all four lineages (Kedinger et al., 1986). Thus, in specific embodiments this source is utilized. The rat intestinal fibroblast cell line F1:G9 has been reported to elicit 3 of the four lineages (all except Paneth cells) when grafted with fetal intestinal endoderm (Fritsch et al., 1997), and in particular embodiments is utilized with the inventive isolated cell preparations.

### Example 9

### Models of Crypt Damage

This example elaborates on the exemplary models referred to at least in Example 6.

A mouse model is developed with local, limited sterilization of intestinal crypts to generate an ideal region for subsequent transplantation of epithelial stem cells. In specific embodiments, an animal model is developed in which a portion of the endogenous stem cells were ablated but the underlying basement membrane, mesenchyme and other components of the stem cell niche remained intact. In a specific embodiment of the present invention, such a model is beneficial to test sternness in the preparations of putative IESCs described herein because it would provide the greatest likelihood that introduced cells would engraft and subsequently demonstrate their capacity to both proliferate and differentiate. Cytotoxic drugs that are known to be capable of destroying both stem cells and clonogenic cells within the small intestinal crypts without affecting the matrix and underlying mesenchyme (Der et al., 1975; Wright et al., 1989; Ijiri and Potten, 1983; Anilkumar et al., 1992; Okudela et al., 1999; Al Dewachi et al., 1980) were utilized. In specific embodiments, this favors both engraftment and differentiation following transplantation of stem cells. In the embodiment wherein stem cells are drawn to the crypt base by chemotaxis, a further advantage of a damage model that leaves the matrix intact is that chemotactic migration of intestinal epithelial cells has recently been shown to be matrix dependent (Polk and Tong, 1999).

The choice of cytotoxic agent was based on the extensive literature available on the effects of chemotherapeutic drugs on the crypts of the mouse small intestine following intraperitoneal (IP) administration (Al Dewachi et al., 1980; Ijiri and Potten, 1987; Moore, 1985). Amongst the group of cytotoxic drugs known to have maximal effects in the lower crypt (Ijiri and Potten, 1987), the exemplary doxorubicin (DOX) and Bleomycin were characterized, because both show very rapid action following IP administration, leading to complete cessation of mitosis within 3 hr (Ijiri and Potten, 1987).

Model I: Local Damage via Luminal Delivery of Cytotoxic Drugs

A surgical model was developed that is simple and rapid enough to allow appropriate numbers of animals to be studied in a reasonable time period. To this end, a procedure was developed in which a 1 cm piece of jejunum is flushed and then clamped (with microvascular serrefines), the drug or vehicle was delivered to the clamped segment via a 27 gauge needle, the bowel is returned to the abdominal cavity and the incisions in both the peritoneum and the skin are sutured closed. The animal was allowed to regain consciousness for 4-6 hr. At the end of this time there was another brief isofluorane anesthesia, during which the original incisions are opened and the clamps are removed. In addition, at this time the location of the clamps was indicated by a piece of 5-0 silk.

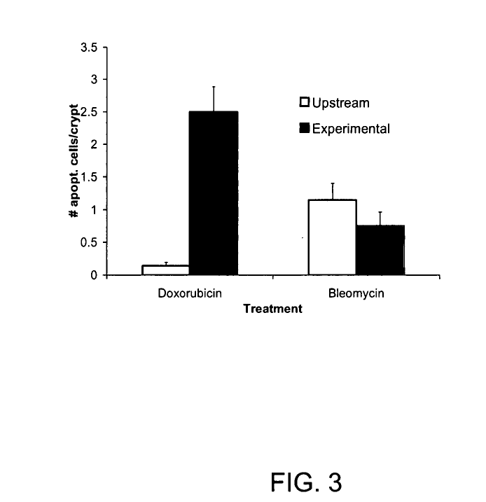

Various doses of DOX and Bleomycin were delivered to clamped segments for 4 hr, then tissue from the experimental segment as well as a segment immediately upstream was collected for histology. Tissue sections were stained with H & E, and apoptosis was assessed by the presence of apoptotic bodies. For quantifying apoptotic cells, 10 crypts per animal (n=3) were selected and scored by a skilled artisan blinded to the experimental conditions. The results at 20 mg/kg BW are shown in FIG. 3. DOX induced significant apoptosis in experimental segments with minimal effects in upstream segments. In contrast, Bleomycin caused apoptosis in both experimental segment and upstream segments. Thus, Bleomycin was being absorbed into the circulation and, thus, affecting the entire intestine. In contrast, DOX appears to be capable of local action, thus creating a suitable model for subsequent experiments.

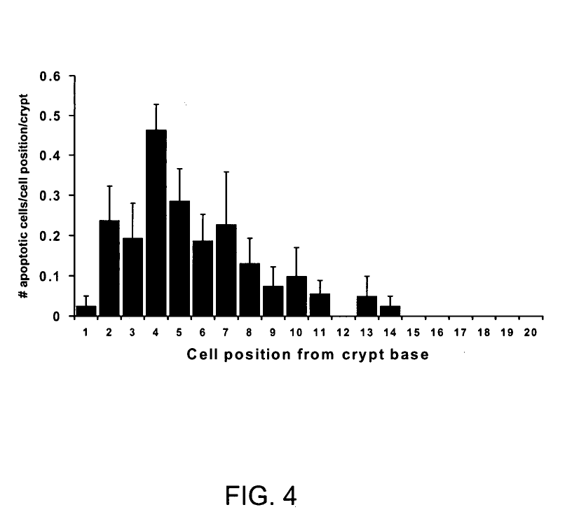

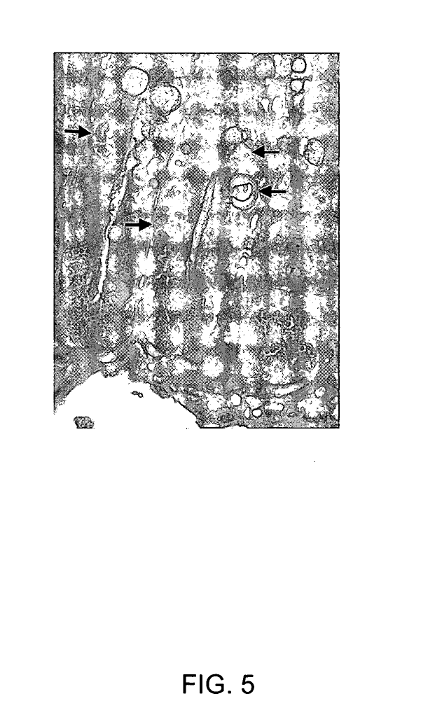

In order to verify that luminally delivered DOX was affecting the stem cell region, the position of apoptotic cells within the crypt was scored. The results (FIG. 4) showed greatest apoptosis in cell positions 4 and 5 that is known as the location of the IESCs (Cheng and Leblond, 1974; Wright, 2000; Booth and Potten, 2000). This is very similar to the pattern that has been previously reported following IP delivery of DOX (Ijiri and Potten, 1987). Careful examination of DOX damaged tissue at both the light (FIG. 5) and EM levels shows no evidence of damage to the basement membrane or to the underlying stromal tissue.

The mice tolerated this clamping procedure extremely well. Following an initial loss of body weight, there is a steady return. Current survival figures for all time points out to 10 days are 95% (77/81). Therefore, this is an excellent model for IV delivery of either bone marrow stem cells or intestinal SP cells, for example. For all of these studies, the fact that only a very small region of the small intestine has been subjected to damage is a critical feature, and assuming that circulating stem cells will home to this region, the extent of engraftment should be much greater than would be the case if the entire intestine were damaged.

Model II: Global Crypt Damage via IP Delivery of DOX

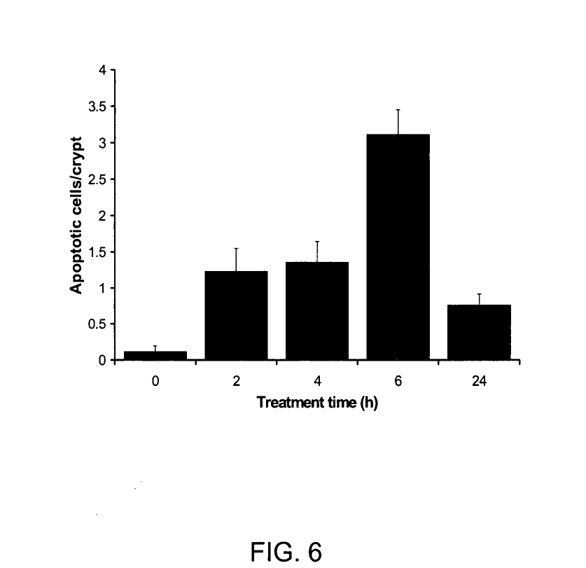

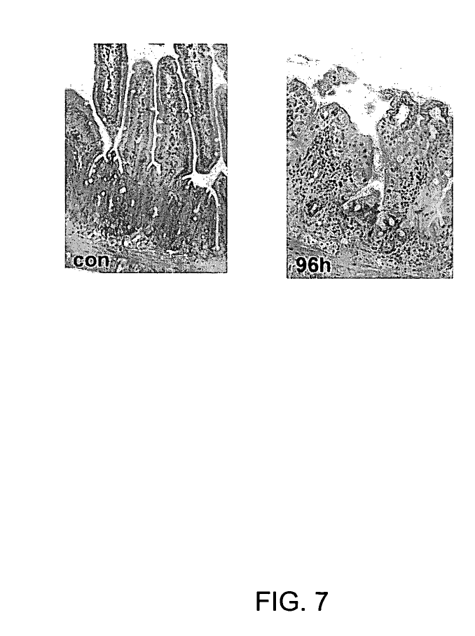

The following model is preferable for luminal delivery of stem cell preparations, particularly wherein a second clamping procedure, such as the day after the damage, is preferable. As there are already published studies on IP delivery of DOX to mice (Ijiri and Potten, 1987; Moore, 1985), minimal additional studies were needed to establish this model. However, although it was already known that an IP dose of 20 mg/kg/BW DOX elicits apoptosis predominantly in the stem cell zone (Ijiri and Potten, 1987) and ablates approximately 50% of the stem cells (Moore, 1985), a complete time course of apoptosis has not been reported. Considering the nature of the timing of stem cell delivery, an apoptosis time course was performed in FIG. 6. As can be seen, apoptosis peeks at 6 hr and is greatly reduced by 24 hr. In contrast, gross morphologic damage is not seen until 72 hr and appears maximal by 96 hr (FIG. 7). By 120 hr the beginning of crypt regeneration from the remaining stem cells is apparent (not shown). Based on these findings, after DOX the optimal time for luminal delivery of stem cells is 6 hr. As the half-life of DOX is 35 min (Lundgren-Eriksson et al., 1997), by 6 hr there should be negligible amounts of the drug remaining in the circulation, and thus no adverse effect would occur on the introduced stem cells. At the same time, a significant proportion of the endogenous stem cells have been ablated, creating an opportunity for the exogenous cells to engraft. The clamping procedure after IP delivery of DOX has been performed successfully, and no evidence was found of gross bleeding into the clamped segment. There is, however, significant morbidity after the IP DOX. For example, body weights initially fall to a similar extent to that after luminal DOX, but in the case of IP DOX they never recover. Although our 10-day survival data after IP DOX is 100%, the literature predicts 40-50% mortality over the next 2 weeks (Oredipe et al., 2003). However, if the myelosuppressive effects of DOX are countered by administration of the drug Swainsonine (Sigma-Aldrich), for example the mice can be maintained until at least 70 days with only 36% mortality (Oredipe et al., 2003).

Based on the literature for IP DOX in mice (Moore, 1985), in specific embodiments the dose of 20 mg/kg BW effectively sterilizes approximately 50% of small intestinal crypts. Moreover, since the quantitation of apoptosis shows essentially the same peak level in Model I after luminal delivery of DOX (FIG. 3 vs. FIG. 6), in specific embodiments approximately 50% of crypts are sterilized within the clamped segment of both models.

### Example 10

### Additional Exemplary Methods and Materials

In some aspects of the invention, the following methods or standard modifications thereof are utilized.

RNA Extraction and Real Time RT-PCR

In some embodiments of the invention, a marker is assayed for in a potential intestinal stem cell by utilizing analysis of RNA, such as by RT-PCR. RNA may be extracted from cell fractions using, for example, the RNeasy Mini Kit from Qiagen. This kit is based on the method of Zimmerman and Shultz (1994) and can be used with as few as 200 cells. It gives good quality RNA from cultured intestinal cells, and, provided the DNase step is included, this RNA is suitable for quantitative RT-PCR. In studies, an estimation for recovery of total epithelial cells is about less than 10%, which provides ample RNA for use in real time RT-PCR. For this purpose, 50 ng of total RNA may be used in a first strand reaction with oligo-DT primers and Superscript reverse transcriptase (Gibco BRL). Aliquots of the resultant cDNA may be used for multiplex real time RT-PCR using specific primers for the stem cell markers and lineage markers listed earlier. Although quantitative data has previously been generated by the inventor and others with regular PCR (Jacomino et al., 1996), accurately detecting differences as low as three-fold, this classical approach becomes cumbersome when applied to a large number of different transcripts, because the conditions (in particular, the number of cycles) for each primer pair have to be adjusted so as to be within the linear range. The great advantage of real time PCR is that the products from every primer pair are automatically “seen” within the linear range. An exemplary ABI 7700 instrument may be utilized, which uses dual fluorogenic probes (Lee et al., 1993), thus allowing a reference mRNA species (e.g. β-actin or GAPDH, for example) to be quantitated in the same tube as each experimental mRNA. In addition, in order to relate results with cell fractions to known levels of expression of these transcripts in the intact intestine, a pooled “standard” RNA is prepared using total RNA from entire jejunum of six adult C57B1/6J mice. An aliquot of this standard is included in every batch of RT-PCR analysis.

As noted earlier, primers for some of the marker mRNAs have been published (Darmoul and Ouellette, 1996; Matsuoka et al., 1999; Macauley et al., 1997). If these prove unsuitable for the universal thermocycling conditions of the ABI 7700 system, alternate primers are designed based on published cDNA sequences and using software provided by the ABI manufacturer, for example. To preclude amplification from trace amounts of contaminating DNA, the forward primer is placed over a splice site (and thus cannot anneal to intronic sequence at its 3′ end). If suitable primers cannot be designed in such locations, the alternate approach is for primers to span a large intron (which would show little amplification due to short cycle times). Either way, the behavior of all primers is checked on mouse genomic DNA, and if any signal is obtained, experimental samples are run with and without reverse transcriptase (RT).

Breeding of ROSA 26 Mice

Breeding pairs of ROSA 26/C57B1/6J(±) mice are available from the Goodell lab. As ROSA (+/+) on this background do not breed well, instead the heterozygotes are mated and the ROSA(+/+) and ROSA(±) offspring are identified by X-gal staining of tissue obtained by ear punch. Both the +/+ and ± males arising from these matings are used as donors because 1 allele of β-gal is sufficient to give strong X-gal staining (Wice and Gordon, 1998). In specific embodiment, the need for heterozygote matings is obviated, such as by using the ROSAs on a mixed C57/129Sv background, which breed well even as homozygotes. In a specific embodiment, a host strain with precisely the same mixed background is utilized. Thus, F1 or F2 animals from C57/129SV crosses as hosts for transplantation experiments may be employed.

Thus, in particular aspects of the invention, the ROSA 26 strain, whose intestinal cells are known to stain positive by a particular technique, are used as donors for the preparation of putative intestinal stem cells. A second set of animals (C67B1/6), whose intestine is negative for the stain, re used as host animals to test whether the putative stem cells can indeed repopulate and restore the intestinal lining. Prior to transplantation, the host animals are treated with a drug that is commonly used in cancer chemotherapy and is known to destroy the dividing cells of the intestinal lining. At various times after transplantation, intestines are harvested from the host mice and are stained to determine the presence of the transplanted cells, and they are also studied to determine whether normal intestinal functions have been restored.

### Example 11

### Therapeutic Applications

This invention relates to the use of stem cells from the gastrointestinal tract and other tissues for engraftment into a damaged region of the gastrointestinal tract. The stem cells may or may not be subjected to genetic manipulation prior to transplantation.

With respect to ultimate therapeutic potential, the ability to rejuvenate the intestinal epithelium by stem cell transplantation has a wide variety of applications in pathological conditions of the small bowel. The first is homologous transplantation in which healthy stem cells could be isolated from one portion of the gut for transplantation into the diseased region. Examples of this application might include radiation and chemotherapy-induced enteritis (where the healthy stem cells are isolated before the patient underwent treatment), necrotizing enterocolitis, small bowel infarction and small bowel injury due to trauma. Other examples include therapy for an individual being treated with or having been treated with nonsteroidal anti-inflammatory drugs, such as those used for pain, arthritis, and so forth, which may cause gastrointestinal damage, including ulcers. The second general scenario is heterologous transplantion in which stem cells harvested from a healthy individual would be used for transplantation into a patient with damaged or diseased bowel. Examples of this application would include various genetic deficiencies of the epithelium, such as SGLT1 deficiency (which causes glucose-galactose malabsorption), as well as acquired disorders such as radiation and chemotherapy-induced enteritis.

In addition, the gastrointestinal epithelium constitutes an ideal site for gene therapy approaches both to intestinal disorders and to a variety of genetic, metabolic and endocrine disorders of other tissues. Although attempts of direct gene transfer to intestinal epithelial stem cells in vivo have met with limited success, this can be attributed largely to the difficulty of generating high titers of suitable vectors. In contrast, in vitro gene transfer is generally more efficient and thus either intestinal stem cells or stem cells from other tissues could be subjected to ex vivo genetic manipulation prior to transplantation. There are numerous disorders that would be amenable to such a gene therapy approach. One notable example would be insulin-dependent diabetes. This prediction is based on the recent report that physiologically-appropriate, glucose-regulated insulin secretion can be achieved in enteroendocrine cells of the intestinal epithelium. Each of the four intestinal epithelial lineages offers unique advantages for various types of gene therapy. Other examples would include: a) the use of secretory cells mucosal vaccination; and b) the use of absorptive cells for gene therapy approaches to acquired diseased such as inflammatory bowel disease (IBD), colon cancer, and for genetic diseases such as phenylketonuria.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PATENTS

U.S. Pat. No.5,786,340

U.S. Pat. No. 5,821,235

U.S. patent application Publication No. US 2004/0058392

### PUBLICATIONS

ABI PRISM 7700 Sequence Detection System. User Bulletin #2. PE Applied Biosystems. 2001.

Abkowitz J L. Can human hematopoietic stem cells become skin, gut, or liver cells? N Engl J Med 2002; 346:770-772.

Akashi K, He X, Chen J, Iwasaki H, Niu C, Steenhard B, Zhang J, Haug J, Li L. Transcriptional accessibility for genes of multiple tissues and hematopoietic lineages is hierarchically controlled during early hematopoiesis. Blood 2003; 101:383-390.

Al Dewachi H S, Wright N A, Appleton D R, Watson A J. The effect of a single injection of cytosine arabinoside on cell population kinetics in the mouse jejunal crypt. Virchows Arch B Cell Pathol Incl Mol Pathol 1980; 34:299-309.

Al Nafussi, A. I. and N. A. Wright. Circadian rhythm in the rate of cellular proliferation and in the size of the functional compartment of mouse jejunal epithelium. Virchows Arch B Cell Pathol. Incl. Mol. Pathol. 40: 71-79, 1982.

Aldhous, P. Stem cells. Panacea, or Pandora's box? Nature 408: 897-898, 2000.

Alison, M. R., R. Poulsom, R. Jeffery, A. P. Dhillon, A. Quaglia, J. Jacob, M. Novelli, G. Prentice, J. Williamson, and N. A. Wright. Hepatocytes from non-hepatic adult stem cells. Nature 406: 257, 2000.

Alvi A J, Clayton H, Joshi C, Enver T, Ashworth A, Vivanco M M, Dale T C, Smalley M J. Functional and molecular characterisation of mammary side population cells. Breast Cancer Res 2003; 5:R1-R8.

Anilkumar T V, Sarraf C E, Hunt T, Alison M R. The nature of cytotoxic drug-induced cell death in murine intestinal crypts. Br J Cancer 1992; 65:552-558.

Asakura A, Seale P, Girgis-Gabardo A, Rudnicki M A. Myogenic specification of side population cells in skeletal muscle. J Cell Biol 2002; 159:123-134.

Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M, Sherlock G. Gene ontology: tool for the unification of biology. The Gene Ontology Consortium. Nat Genet 2000; 25:25-29.

Bailey T L, Gribskov M. Methods and statistics for combining motif match scores. J Comput Biol 1998; 5:211-221.

Balda M S, Whitney J A, Flores C, Gonzalez S, Cereijido M, Matter K. Functional dissociation of paracellular permeability and transepithelial electrical resistance and disruption of the apical-basolateral intramembrane diffusion barrier by expression of a mutant tight junction membrane protein. J Cell Biol 1996; 134:1031-1049.

Batlle E, Henderson J T, Beghtel H, van den Born M M, Sancho E, Huls G, Meeldijk J, Robertson J, van de W M, Pawson T, Clevers H. Beta-catenin and TCF mediate cell positioning in the intestinal epithelium by controlling the expression of EphB/ephrinB. Cell 2002; 11 1:251-263.

Ben Arie, N., B. A. Hassan, N. A. Bermingham, D. M. Malicki, D. Armstrong, M. Matzuk, H. J. Bellen, and H. Y. Zoghbi. Functional conservation of atonal and Math1 in the CNS and PNS. Development 127: 1039-1048, 2000.

Ben Arie, N., H. J. Bellen, D. L. Armstrong, A. E. McCall, P. R. Gordadze, Q. Guo, M. M. Matzuk, and H. Y. Zoghbi. Math1 is essential for genesis of cerebellar granule neurons. Nature 390: 169-172, 1997.

Bens M, Bodganova A, Cluzeaud F, Miquerol L, Kerneis S, Kraehenbuhl J P, Kahn A, Pringault E, Vandewalle A. Transimmortalized mouse intestinal cells (m-IC_{c12}) that maintain a crypt phenotype. Am J Physiol 1996; 270:1666-1674.

Bens, M., A. Bodganova, F. Cluzeaud, L. Miquerol, S. Kerneis, J. P. Kraehenbuhl, A. Kahn, E. Pringault, and A. Vandewalle. Transimmortalized mouse intestinal cells (m-ICc12) that maintain a crypt phenotype. Am. J. Physiol. 270: 1666-1674, 1996.

Bermingham, N. A., B. A. Hassan, S. D. Price, M. A. Vollrath, N. Ben Arie, R. A. Eatock, H. J. Bellen, A. Lysakowski, and H. Y. Zoghbi. Math1: an essential gene for the generation of inner ear hair cells. Science 284: 1837-1841, 1999.

Bittner, R. E., C. Schofer, K. Weipoltshammer, S. Ivanova, B. Streubel, E. Hauser, M. Freilinger, H. Hoger, A. Elbe-Burger, and F. Wachtler. Recruitment of bone-marrow-derived cells by skeletal and cardiac muscle in adult dystrophic mdx mice. Anat. Embryol. (Berl) 199: 391-396, 1999.

Bjerknes M, Cheng H. Clonal analysis of mouse intestinal epithelial progenitors. Gastroenterology 1999; 116:7-14.

Bjerknes M, Cheng H. Methods for the isolation of intact epithelium from the mouse intestine. Anat Rec 1981; 199:565-574.

Bjerknes M, Cheng H. Modulation of specific intestinal epithelial progenitors by enteric neurons. Proc Natl Acad Sci USA 2001; 98:12497-12502.

Bjornson, C. R., R. L. Rietze, B. A. Reynolds, M. C. Magli, and A. L. Vescovi. Turning brain into blood: a hematopoietic fate adopted by adult neural stem cells in vivo. Science 283: 534-537, 1999.

Booth C, Brady G, Potten C S. Crowd control in the crypt. Nat Med 2002; 8: 1360-1361.

Booth C, Potten C S. Gut instincts: thoughts on intestinal epithelial stem cells. J Clin Invest 2000; 105:1493-1499.

Booth, C., J. A. O'Shea, and C. S. Potten. Maintenance of functional stem cells in isolated and cultured adult intestinal epithelium. Exp. Cell Res. 249: 359-366, 1999.

Brenner, D. A. Bone marrow instead of liver transplantation? Gastroenterology 119: 891-892, 2000.

Brittan M, Hunt T, Jeffery R, Poulsom R, Forbes S J, Hodivala-Dilke K, Goldman J, Alison M R, Wright N A. Bone marrow derivation of pericryptal myofibroblasts in the mouse and human small intestine and colon. Gut 2002; 50:752-757.

Brittan M, Wright N A. Gastrointestinal stem cells. J Pathol 2002; 197:492-509.

Bunting, K. D., S. Zhou, T. Lu, and B. P. Sorrentino. Enforced P-glycoprotein pump function in murine bone marrow cells results in expansion of side population stem cells in vitro and repopulating cells in vivo. Blood 96: 902-909, 2000.

Bussemaker H J, Li H, Siggia E D. Regulatory element detection using correlation with expression. Nat Genet 2001; 27:167-171.

Chandrasena, G., I. Sunitha, C. Lau, N. N. Nanthakumar, and S. J. Henning. Expression of sucrase-isomaltase mRNA along the villus-crypt axis in the rat small intestine. Cell. Mol. Biol. 38: 243-254, 1992.

Chang W W, Leblond C P. Renewal of the epithelium in the descending colon of the mouse. I. Presence of three cell populations: vacuolated-columnar, mucous and argentaffin. Am J Anat 1971; 131:73-99.

Cheng D Y, Kolls J K, Lei D, Noel R A. In vivo and in vitro gene transfer and expression in rat intestinal epithelial cells by E1-deleted adenoviral vector. Hum Gene Ther 1997; 8:755-764.

Cheng H, Bjerknes M. Whole population cell kinetics and postnatal development of the mouse intestinal epithelium. Anat Rec 1985; 211:420-426.

Cheng H, Leblond C P. Origin, differentiation and renewal of the four main epithelial cell types in the mouse small intestine. V. Unitarian theory of the origin of the four epithelial cell types. Am J Anat 1974; 141 :537-562.

Cheng, H. and M. Bjerknes. Cell production in mouse intestinal epithelium measured by stathmokinetic flow cytometry and Coulter particle counting. Anat. Rec. 207: 427-434, 1983.

Cheng, H., M. Bjerknes, and H. Chen. CRP-ductin: a gene expressed in intestinal crypts and in pancreatic and hepatic ducts. Anat. Rec. 244: 327-343, 1996.

Cheung A T, Dayanandan B, Lewis J T, Korbutt G S, Rajotte R V, Bryer-Ash M, Boylan M O, Wolfe M M, Kieffer T J. Glucose-dependent insulin release from genetically engineered K cells. Science 2000; 290:1959-1962.

Clatworthy J P, Subramanian V, V. Stem cells and the regulation of proliferation, differentiation and patterning in the intestinal epithelium: emerging insights from gene expression patterns, transgenic and gene ablation studies. Mech Dev 2001; 101:3-9.

Cohn S M, Schloemann S, Tessner T, Seibert K, Stenson W F. Crypt stem cell survival in the mouse intestinal epithelium is regulated by prostaglandins synthesized through cyclooxygenase-1. J Clin Invest 1997; 99:1367-1379.

Cooper H S, Murthy S N, Shah R S, Sedergran D J. Clinicopathologic study of dextran sulfate sodium experimental murine colitis. Lab Invest 1993; 69:238-249.

Cosentino L, Shaver-Walker P, Heddle J A. The relationships among stem cells, crypts, and villi in the small intestine of mice as determined by mutation tagging. Dev Dyn 1996; 207:420-428.

Croop, J. M., M. Raymond, D. Haber, A. Devault, R. J. Arceci, P. Gros, and D. E. Housman. The three mouse multidrug resistance (mdr) genes are expressed in a tissue-specific manner in normal mouse tissues. Mol. Cell Biol. 9: 1346-1350, 1989.

Daley G Q, Goodell M A, Snyder E Y. Realistic prospects for stem cell therapeutics. Hematology (Am Soc Hematol Educ Program) 2003; 398-418.

Darmoul, D. and A. J. Ouellette. Positional specificity of defensin gene expression reveals Paneth cell heterogeneity in mouse small intestine. Am. J. Physiol. 271: G68-G74, 1996.

De Geus, B., E. M. Van den, C. Coolen, L. Nagelkerken, H. P. Van der, and J. Rozing. Phenotype of intraepithelial lymphocytes in euthymic and athymic mice: implications for differentiation of cells bearing a CD3-associated gamma delta T cell receptor. Eur. J. Immunol. 20: 291-298, 1990.

Del Buono R, Fleming K A, Morey A L, Hall P A, Wright N A. A nude mouse xenograft model of fetal intestine development and differentiation. Development 1992; 114:67-73.

Der S, Jonek J J, Konecki J. The effect of 5-fluorouracil on the mitotic cycle and DNA synthesis in the epithelium of mouse small intestine. Folia Histochem Cytochem (Krakow) 1975; 13:21-35.

DiBiase M D, Rhodes C T. Investigations of epidermal growth factor in semisolid formulations. Pharm Acta Helv 1991; 66:165-169.

Dobbins W O, III, Austin L L. Electron microscopic definition of intestinal endocrine cells: immunogold localization and review. Ultrastruct Pathol 1991; 15:15-39.

Drucker D J. Glucagon-like peptides: regulators of cell proliferation, differentiation, and apoptosis. Mol Endocrinol 2003; 17:161-171.

Duluc I, Freund J-N, Leberquier C, Kedinger M. Fetal endoderm primarily holds the temporal and positional information required for mammalian intestinal development. J Cell Biol 1994; 126:211-221.

Dvorak B, Halpern M D, Holubec H, Williams C S, McWilliam D L, Dominguez J A, Stepankova R, Payne C M, McCuskey R S. Epidermal growth factor reduces the development of necrotizing enterocolitis in a neonatal rat model. Am J Physiol Gastrointest Liver Physiol 2002; 282:G156-G164.

Eberwine J, Yeh H, Miyashiro K, Cao Y, Nair S, Finnell R, Zettel M, Coleman P. Analysis of gene expression in single live neurons. Proc Natl Acad Sci USA 1992; 89:3010-3014.

Eckert J W, Buerkle C J, Major A M, Finegold M J, Brandt M L. In situ hybridization utilizing a Y chromosome DNA probe. Use as a cell marker for hepatocellular transplantation. Transplantation 1995; 59:109-111.

Eglitis, M. A. and E. Mezey. Hematopoietic cells differentiate into both microglia and macroglia in the brains of adult mice. Proc. Natl. Acad. Sci. U.S.A 94: 4080-4085, 1997.

Evans G S, Flint N, Potten C S. Primary cultures for studies of cell regulation and physiology in intestinal epithelium. Ann Rev Physiol 1994; 56:399-417.

Evans G S, Flint N, Somers A S, Eyden B, Potten C S. The development of a method for the preparation of rat intestinal epithelial cell primary cultures. J Cell Sci 1992; 101:219-231.

Evans R A, Tian Y C, Steadman R, Phillips A O. TGF-beta1-mediated fibroblast-myofibroblast terminal differentiation-the role of Smad proteins. Exp Cell Res 2003; 282:90-100.

Ferguson A, Gerskowitch V P, Russell R I. Pre- and postweaning disaccharidase patterns in isografts of fetal mouse intestine. Gastroenterology 1973 ; 64:292-297.

Ferrari, G., G. Cusella-De Angelis, M. Coletta, E. Paolucci, A. Stornaiuolo, G. Cossu, and F. Mavilio. Muscle regeneration by bone marrow-derived myogenic progenitors. Science 279: 1528-1530, 1998.

Ferretti E, Li S, Wang J, Post M, Moore A. Mesenchymal regulation of differentiation of intestinal epithelial cells. J Pediatr Gastroenterol Nutr 1996; 23:65-73.

Fiering, S. N., M. Roederer, G. P. Nolan, D. R. Micklem, D. R. Parks, and L. A. Herzenberg. Improved FACS-Gal: flow cytometric analysis and sorting of viable eukaryotic cells expressing reporter gene constructs. Cytometry 12: 291-301, 1991.

Forbes S J, Hodgson H J. Review article: gene therapy in gastroenterology and hepatology. Aliment Pharmacol Ther 1997; 11:823-836.

Foreman P K, Wainwright M J, Alicke B, Koveski I, Wickham T J, Smith J G, Meier-Davis S, Fix J A, Daddona P, Gardner P, Huang M T F. Adenovirus-mediated transduction of intestinal cells in vivo. Hum Gene Ther 1998; 9:1313-1321.

Fortunel N O, Otu H H, Ng H H, Chen J, Mu X, Chevassut T, Li X, Joseph M, Bailey C, Hatzfeld J A, Hatzfeld A, Usta F, Vega V B, Long P M, Libermann T A, Lim B. Comment on “‘Stemness’: transcriptional profiling of embryonic and adult stem cells” and “a stem cell molecular signature”. Science 2003; 302:393.

Fowler E B, Cuenin M F, Hokett S D, Peacock M E, McPherson J C, III, Dirksen T R, Sharawy M, Billman M A. Evaluation of pluronic polyols as carriers for grafting materials: study in rat calvaria defects. J Periodontol 2002; 73:191-197.

Fritsch, C., P. Simon-Assmann, M. Kedinger, and G. S. Evans. Cytokines modulate fibroblast phenotype and epithelial-stroma interactions in rat intestine. Gastroenterology 112: 826-838, 1997.

Fujimoto K, Beauchamp R D, Whitehead R H. Identification and isolation of candidate human colonic clonogenic cells based on cell surface integrin expression. Gastroenterology 2002; 123:1941-1948.

Fujimoto, K., Beauchamp, R. D., and Whitehead, R. H. Identification and isolation of candidate human colonic stem cells based on cell surface integrin expression. Gastroenterology 120, A-61. 2001.

Gilroy R, Sudan D. Liver and small bowel transplantation: therapeutic alternatives for the treatment of liver disease and intestinal failure. Semin Liver Dis 2000; 20:437-450.

Good J R, Cabral M, Sharma S, Yang J, Van Driessche N, Shaw C A, Shaulsky G, Kuspa A. TagA, a putative serine protease/ABC transporter of Dictyostelium that is required for cell fate determination at the onset of development. Development 2003; 130:2953-2965.

Goodell M A, Brose K, Paradis G, Conner A S, Mulligan R C. Isolation and functional properties of murine hematopoietic stem cells that are replicating in vivo. Journal of Experimental Medicine 1996; 183:1797-1806.

Goodell M A, Rosenzweig M, Kim H, Marks D F, DeMaria M, Paradis G, Grupp S A, Sieff C A, Mulligan R C, Johnson R P. Dye efflux studies suggest that hematopoietic stem cells expressing low or undetectable levels of CD34 antigen exist in multiple species. Nat Med 1997; 3:1337-1345.

Goodman, L. S. and A. Gilman. The pharmacological basis of therapeutics. New York, New York, Macmillan Publishing Co., Inc. 1975.

Goodman, T. and L. Lefrancois. Expression of the gamma-delta T-cell receptor on intestinal CD8+ intraepithelial lymphocytes. Nature 333: 855-858, 1988.

Gordon J I, Schmidt G H, Roth K A. Studies of intestinal stem cells using normal, chimeric, and transgenic mice. FASEB J 1992; 6:3039-3050.

Grand R J, Li W, Montgomery R K, Buller H A. Gene therapy for gastrointestinal disorders: just around the corner? J Pediatr Gastroenterol Nutr 1999; 29:375-376.

Gussoni E, Soneoka Y, Strickland C D, Buzney E A, Khan M K, Flint A F, Kunkel L M, Mulligan R C. Dystrophin expression in the mdx mouse restored by stem cell transplantation. Nature 1999; 401 :390-394.

Hagemann R F, Sigdestad C P, Lesher S. A quantitative description of the intestinal epithelium of the mouse. Am J Anat 1970; 129:41-51.

Henning S J, Rubin D C, Shulman R J. Ontogeny of the intestinal mucosa. In: Johnson L R, ed. Physiology of the Gastrointestinal Tract. 3rd ed. New York: Raven Press, 1994:571-610.

Henning S J. Gene transfer into the intestinal epithelium. Adv Drug Del Rev 1995; 17:341-347.

Ho S B, Itzkowitz S H, Friera A M, Jiang S H, Kim Y S. Cell lineage markers in premalignant and malignant colonic mucosa. Gastroenterology 1989; 97:392-404.

Holden C, Vogel G. Stem cells. Plasticity: time for a reappraisal? Science 2002; 296:2126-2129.

Horwitz, E. M., D. J. Prockop, L. A. Fitzpatrick, W. W. Koo, P. L. Gordon, M. Neel, M. Sussman, P. Orchard, J. C. Marx, R. E. Pyeritz, and M. K. Brenner. Transplantability and therapeutic effects of bone marrow-derived mesenchymal cells in children with osteogenesis imperfecta. Nat. Med 5: 309-313, 1999.

Houchen C W, George R J, Sturmoski M A, Cohn S M. FGF-2 enhances intestinal stem cell survival and its expression is induced after radiation injury. Am J Physiol 1999; 39:G249-G258.

Housley R M, Morris C F, Boyle W, Ring B, Biltz R, Tarpley J E, Aukerman S L, Devine P L, Whitehead R H, Pierce G F. Keratinocyte growth factor induces proliferation of hepatocytes and epithelial cells throughout the rat gastrointestinal tract. J Clin Invest 1994; 94:1764-1777.

Ijiri K, Potten C S. Further studies on the response of intestinal crypt cells of different hierarchical status to eighteen different cytotoxic agents. Br J Cancer 1987; 55:113-123.

Ijiri K, Potten C S. Response of intestinal cells of differing topographical and hierarchical status to ten cytotoxic drugs and five sources of radiation. Br J Cancer 1983; 47:175-185.

Ikuta, K. and I. L. Weissman. Evidence that hematopoietic stem cells express mouse c-kit but do not depend on steel factor for their generation. Proc. Natl. Acad. Sci. U.S.A 89: 1502-1506, 1992.

Irizarry R A, Hobbs B, Collin F, Beazer-Barclay Y D, Antonellis K J, Scherf U, Speed T P. Exploration, normalization, and summaries of high density oligonucleotide array probe level data. Biostatistics 2003; 4:249-264.

Ivanova N B, Dimos J T, Schaniel C, Hackney J A, Moore K A, Lemischka I R. A stem cell molecular signature. Science 2002; 298:601-604.

Jackson K A, Majka S M, Wang H, Pocius J, Hartley C J, Majesky M W, Entman M L, Michael L H, Hirschi K K, Goodell M A. Regeneration of ischemic cardiac muscle and vascular endothelium by adult stem cells. J Clin Invest 2001; 107:1395-1402.

Jackson K A, Mi T, Goodell M A. Hematopoietic potential of stem cells isolated from murine skeletal muscle. Proc Natl Acad Sci USA 1999; 96:14482-14486.

Jacomino M, Lau C, James S Z, Shukla P, Henning S J. Gene transfer into fetal rat intestine. Hum Gene Ther 1996; 7:1757-1762.

Jacomino M, Shukla P, Henning S J. Use of amphotropic retroviral vectors for gene transfer in human colon carcinoma cells. Hum Gene Ther 1997; 8:835-841.

Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M. Pluripotency of mesenchymal stem cells derived from adult marrow. Nature 2002; 418:41-49.

Jones M K, Kawanaka H, Baatar D, Szabo I L, Tsugawa K, Pai R, Koh G Y, Kim I, Sarfeh I J, Tamawski A S. Gene therapy for gastric ulcers with single local injection of naked DNA encoding VEGF and angiopoietin-1. Gastroenterology 2001; 121:1040-1047.

Kabanov A V, Batrakova E V, Alakhov V Y. Pluronic block copolymers as novel polymer therapeutics for drug and gene delivery. J Control Release 2002; 82:189-212.

Kaestner K H, Silberg D G, Traber P G, Schütz G. The mesenchymal winged helix transcription factor Fkh6 is required for the control of gastrointestinal proliferation and differentiation. Genes Dev 1997; 11:1583-1595.

Kaihara S, Kim S, Benvenuto M, Kim B S, Mooney D J, Tanaka K, Vacanti J P. End-to-end anastomosis between tissue-engineered intestine and native small bowel. Tissue Eng 1999; 5:339-346.

Kaihara S, Kim S S, Kim B S, Mooney D, Tanaka K, Vacanti J P. Long-term follow-up of tissue-engineered intestine after anastomosis to native small bowel. Transplantation 2000; 69:1927-1932.

Kaiserlian, D., C. Roche, M. Blanc, and J. P. Revillard. A rapid mechanical vibration procedure for purifying mature (Ia positive) columnar epithelial cells from the mouse small intestine. J Immunol. Methods 122: 243-252, 1989.

Kaufman S S, Atkinson J B, Bianchi A, Goulet O J, Grant D, Langnas A N, McDiarmid S V, Mittal N, Reyes J, Tzakis A G. Indications for pediatric intestinal transplantation: a position paper of the American Society of Transplantation. Pediatr Transplant 2001 ; 5:80-87.

Kawaguchi A L, Dunn J C, Fonkalsrud E W. In vivo growth of transplanted genetically altered intestinal stem cells. J Pediatr Surg 1998; 33:559-563.

Kayahara T, Sawada M, Takaishi S, Fukui H, Seno H, Fukuzawa H, Suzuki K, Hiai H, Kageyama R, Okano H, Chiba T. Candidate markers for stem and early progenitor cells, Musashi-1 and Hes1, are expressed in crypt base columnar cells of mouse small intestine. FEB S Lett 2003; 535:131-135.

Kedinger M, Haffen K, Simon-Assmann P. Intestinal tissue and cell cultures. Differentiation 1987; 36:71-85.

Kedinger M, Simon-Assmann P, Alexandre E, Haffen K. Importance of a fibroblastic support for in vitro differentiation of intestinal endodermal cells and for their response to glucocorticoids. Cell Differ 1987; 20:171-182.

Kedinger M, Simon-Assmann P M, Lacroix B, Marxer A, Hauri H P, Haffen K. Fetal gut mesenchyme induces differentiation of cultured intestinal endodermal and crypt cells. Dev Biol 1986; 13:474-483.

Kedinger, M., K. Haffen, and P. Simon-Assmann. Intestinal tissue and cell cultures. Differentiation 36: 71-85, 1987.

Kedinger, M., P. M. Simon-Assmann, B. Lacroix, A. Marxer, H. P. Hauri, and K. Haffen. Fetal gut mesenchyme induces differentiation of cultured intestinal endodermal and crypt cells. Dev. Biol. 113: 474-483, 1986.

Kedinger, M., P. Simon-Assmann, E. Alexandre, and K. Haffen. Importance of a fibroblastic support for in vitro differentiation of intestinal endodermal cells and for their response to glucocorticoids. Cell Differ. 20: 171-182, 1987.

Kibler K, Nguyen T L, Svetz J, Van Driessche N, Ibarra M, Thompson C, Shaw C, Shaulsky G. A novel developmental mechanism in Dictyostelium revealed in a screen for communication mutants. Dev Biol 2003; 259:193-208.

Kocher A A, Schuster M D, Szabolcs M J, Takuma S, Burkhoff D, Wang J, Homma S, Edwards N M, Itescu S. Neovascularization of ischemic myocardium by human bone-marrow-derived angioblasts prevents cardiomyocyte apoptosis, reduces remodeling and improves cardiac function. Nat Med 2001; 7:430-436.

Korbling M, Katz R L, Khanna A, Ruifrok A C, Rondon G, Albitar M, Champlin R E, Estrov Z. Hepatocytes and epithelial cells of donor origin in recipients of peripheral-blood stem cells. N Engl J Med 2002; 346:738-746.

Korinek V, Barker N, Moerer P, van Donselaar E, Huls G, Peters P J, Clevers H. Depletion of epithelial stem-cell compartments in the small intestine of mice lacking Tcf-4. Nat Genet 1998; 19:379-383.

Kozar R A, Schultz S G, Hassoun H T, Desoignie R, Weisbrodt N W, Haber M M, Moore F A. The type of sodium-coupled solute modulates small bowel mucosal injury, transport function, and ATP after ischemia/reperfusion injury in rats. Gastroenterology 2002; 123:810-816.

Krause D S, Theise N D, Collector M I, Henegariu O, Hwang S, Gardner R, Neutzel S, Sharkis S J. Multi-organ, multi-lineage engraftment by a single bone marrow-derived stem cell. Cell 2001; 105:369-377.

Lagasse E, Connors H, Al-Dhalimy M, Reitsma M, Dohse M, Osborne L, Wang X, Finegold M, Weissman I L, Grompe M. Purified hematopoietic stem cells can differentiate into hepatocytes in vivo. Nature Med 2000; 6:1229-1234.

Lam M M, O'Connor T P, Diamond J. Loads, capacities and safety factors of maltase and the glucose transporter SGLT1 in mouse intestinal brush border. J Physiol 2002; 542:493-500.

Lau C, Soriano H E, Ledley F D, Finegold M J, Wolfe J H, Birkenmeier E H, Henning S J. Retroviral gene transfer into the intestinal epithelium. Hum Gene Ther 1995; 6:1145-1151.

Lee, L. G., C. R. Connell, and W. Bloch. Allelic discrimination by nick-translation PCR with fluorogenic probes. Nucl. Acids Res. 21: 3761-3766, 1993.

Lenhard B, Sandelin A, Mendoza L, Engstrom P, Jareborg N, Wasserman W W. Identification of conserved regulatory elements by comparative genome analysis. J Biol 2003; 2:13.

Li, M., R. Hurren, R. L. Zastawny, V. Ling, and R. N. Buick. Regulation and expression of multidrug resistance (MDR) transcripts in the intestinal epithelium. Br. J Cancer 80: 1123-1131, 1999.

Liu X, Brutlag D L, Liu J S. BioProspector: discovering conserved DNA motifs in upstream regulatory regions of co-expressed genes. Pac Symp Biocomput 2001; 127-138.

Lundgren-Eriksson L, Carlsson A, Eksborg S, Ryd W, Vesanen R, Hultborn R. Pharmacokinetics of doxorubicin and epirubicin in mice during chlorpromazine-induced hypothermia. Cancer Chemother Pharmacol 1997; 40:419-424.

Macauley, S. P., R. W. Tarnuzzer, G. S. Schultz, N. Chegini, G. E. Oxford, and M. G. Humphreys-Beher. Extracellular-matrix gene expression during mouse submandibular gland development. Arch Oral Biol. 42: 443-454, 1997.

Mahler M, Bristol I J, Leiter E H, Workman A E, Birkenmeier E H, Elson C O, Sundberg J P. Differential susceptibility of inbred mouse strains to dextran sulfate sodium-induced colitis. Am J Physiol 1998; 274:G544-G551.

Mashimo, H., D. K. Podolsky, and M. C. Fishman. Structure and expression of murine intestinal trefoil factor: high evolutionary conservation and postnatal expression. Biochem Biophys. Res. Commun. 210: 31-37, 1995.

Matsuoka, Y., J. C. Pascall, and K. D. Brown. Quantitative analysis reveals differential expression of mucin (MUC2) and intestinal trefoil factor mRNAs along the longitudinal axis of rat intestine. Biochim. Biophys. Acta 1489: 336-344, 1999.

Medvinsky A, Smith A. Stem cells: Fusion brings down barriers. Nature 2003; 422:823-825.

Mills J C, Andersson N, Hong C V, Stappenbeck T S, Gordon J I. Molecular characterization of mouse gastric epithelial progenitor cells. Proc Natl Acad Sci USA 2002; 99:14819-14824.

Mills J C, Gordon J I. The intestinal stem cell niche: There grows the neighborhood. Proc Natl Acad Sci USA 2001; 98:12334-12336.

Montgomery R K, Sybicki M A, Grand R J. Autonomous biochemical and morphological differentiation in fetal rat intestine transplanted at 17 and 20 days of gestation. Dev Biol 1981; 87:76-84.

Montgomery R K. Morphogenesis in vitro of dissociated fetal rat small intestinal cells upon an open surface and subsequent to collagen gel overlay. Dev Biol 1986; 17:64-70.

Moore J V. Clonogenic response of cells of murine intestinal crypts to 12 cytotoxic drugs. Cancer Chemother Pharmacol 1985; 15:11-15.

Musch M W, Clarke L L, Mamah D, Gawenis L R, Zhang Z, Ellsworth W, Shalowitz D, Mittal N, Efthimiou P, Alnadjim Z, Hurst S D, Chang E B, Barrett T A. T cell activation causes diarrhea by increasing intestinal permeability and inhibiting epithelial Na+/K+-APTase. J Clin Invest 2002; 110:1739-1747.

Nalbandian R M, Henry R L, Balko K W, Adams D V, Neuman N R. Pluronic F-127 gel preparation as an artificial skin in the treatment of third-degree burns in pigs. J Biomed Mater Res 1987; 21:1135-1148.

Nanthakumar N N, Henning S J. Distinguishing normal and glucocorticoid-induced maturation of intestine using bromodeoxyuridine. Am J Physiol 1995; 268:G139-G145.

Nishimura S, Wakabayashi N, Toyoda K, Kashima K, Mitsufuji S. Expression of Musashi-1 in human normal colon crypt cells: a possible stem cell marker of human colon epithelium. Dig Dis Sci 2003; 48:1523-1529.

Noel R A, Shukla P, Henning S J. Optimization of gene transfer into intestinal epithelial cells using a retroviral vector. J Pediatr Gastroenterol Nutr 1994; 19:43-49.

O'Brien D P, Nelson L A, Kemp C J, Williams J L, Wang Q, Erwin C R, Hasselgren P O, Warner B W. Intestinal permeability and bacterial translocation are uncoupled after small bowel resection. J Pediatr Surg 2002; 37:390-394.

Oesterreicher T J, Leeper L L, Darlington G J, Henning S J. Mice lacking C/EBPα show normal perinatal expression of various intestinal mRNA's. Gastroenterology 110, A826. 1996.

Oesterreicher, T. J., L. L. Leeper, M. J. Finegold, G. J. Darlington, and S. J. Henning. Intestinal maturation in mice lacking CCAAT/enhancer-binding protein alpha (C/EPBalpha). Biochem J 330 (Pt 3): 1165-1171, 1998.

Ogata H, Goto S, Sato K, Fujibuchi W, Bono H, Kanehisa M. KEGG: Kyoto Encyclopedia of Genes and Genomes. Nucleic Acids Res 1999; 27:29-34.

Oh H, Bradfute S B, Gallardo T D, Nakamura T, Gaussin V, Mishina Y, Pocius J, Michael L H, Behringer R R, Garry D J, Entman M L, Schneider M D. Cardiac progenitor cells from adult myocardium: homing, differentiation, and fusion after infarction. Proc Natl Acad Sci USA 2003; 100:12313-12318.

Okamoto R, Yajima T, Yamazaki M, Kanai T, Mukai M, Okamoto S, Ikeda Y, Hibi T, Inazawa J, Watanabe M. Damaged epithelia regenerated by bone marrow-derived cells in the human gastrointestinal tract. Nat Med 2002; 8:1011-1017.

Okayasu I, Hatakeyama S, Yamada M, Ohkusa T, Inagaki Y, Nakaya R. A novel method in the induction of reliable experimental acute and chronic ulcerative colitis in mice. Gastroenterology 1990; 98:694-702.

Okudela K, Ito T, Mitsui H, Hayashi H, Udaka N, Kanisawa M, Kitamura H. The role of p53 in bleomycin-induced DNA damage in the lung. A comparative study with the small intestine. Am J Pathol 1999; 155:1341-1351.

Olsen L, Hansen M, Ekstrom C T, Troelsen J T, Olsen J. The crypt/villus in situ hybridization database. Bioinformatics In Press. 2004.

Oredipe O A, Furbert-Harris P M, Laniyan I, Green W R, Griffin W M, Sridhar R. Coadministration of swainsonine and doxorubicin attenuates doxorubicin-induced lethality in mice. Cell Mol Biol (Noisy-le-grand) 2003; 49:1037-1048.

Orkin S H. Stem cell alchemy. Nat Med 2000; 6:1212-1213.

Orlic, D., J. Kajstura, S. Chimenti, I. Jakoniuk, S. M. Anderson, B. Li, J. Pickel, R. McKay, B. Nadal-Ginard, D. M. Bodine, A. Leri, and P. Anversa. Bone marrow cells regenerate infarcted myocardium. Nature 410: 701-705, 2001.

Ouellette, A. J. and M. E. Selsted. Paneth cell defensins: Endogenous peptide components of intestinal host defense. FASEB J. 10: 1280-1289, 1996.

Pageot L P, Perreault N, Basora N, Francoeur C, Magny P, Beaulieu J F. Human cell models to study small intestinal functions: recapitulation of the crypt-villus axis. Microsc Res Tech 2000; 49:394-406.

Pallavicini, M. G., C. R. Ng, and J. W. Gray. Bivariate flow cytometric analysis of murine intestinal epithelial cells for cytokinetic studies. Cytometry 5: 55-62, 1984.

Pang G, Buret A, O'Loughlin E, Smith A, Batey R, Clancy R. Immunologic, functional, and morphological characterization of three new human small intestinal epithelial cell lines. Gastroenterology 1996; 111:8-18.

Paris F, Fuks Z, Kang A, Capodieci P, Juan G, Ehleiter D, Haimovitz-Friedman A, Cordon-Cardo C, Kolesnick R. Endothelial apoptosis as the primary lesion initiating intestinal radiation damage in mice. Science 2001 ; 293:293-297.

Parrish, E. P., C. Cifuentes-Diaz, Z. L. Li, P. Vicart, D. Paulin, P. A. Dreyfus, M. Peschanski, A. J. Harris, and L. Garcia. Targeting widespread sites of damage in dystrophic muscle: engrafted macrophages as potential shuttles. Gene Ther. 3: 13-20, 1996.

Patel H R, Tait I S, Evans G S, Campbell F C. Influence of cell interactions in a novel model of postnatal mucosal regeneration. Gut 1996; 38:679-686.

Paul E C A, Hochman J, Quaroni A. Conditionally immortalized intestinal epithelial cells: novel approach for study of differentiated enterocytes. Am J Physiol 1993; 265:C266-C278.

Perreault, N. and J. F. Beaulieu. Primary cultures of fully differentiated and pure human intestinal epithelial cells. Exp. Cell Res. 245: 34-42, 1998.

Perret V, Lev R, Pigman W. Simple method for the preparation of single cell suspensions from normal and tumorous rat colonic mucosa. Gut 1977; 18:382-385.

Petersen, B. E., W. C. Bowen, K. D. Patrene, W. M. Mars, A. K. Sullivan, N. Murase, S. S. Boggs, J. S. Greenberger, and J. P. Goff. Bone marrow as a potential source of hepatic oval cells. Science 284: 1168-1170, 1999.

Polk D B, Tong W. Epidermal and hepatocyte growth factors stimulate chemotaxis in an intestinal epithelial cell line. Am J Physiol 1999; 277:C1149-C1159.

Potten C S, Booth C, Tudor G L, Booth D, Brady G, Hurley P, Ashton G, Clarke R, Sakakibara S, Okano H. Identification of a putative intestinal stem cell and early lineage marker; musashi-1. Differentiation 2003; 71:28-41.

Potten C S, Loeffler M. Stem cells: attributes, cycles, spirals, pitfalls and uncertainties. Lessons for and from the Crypt. Development 1990; 110:1001-1020.

Potten C S. Stem cells in gastrointestinal epithelium: numbers, characteristics and death. Philos Trans R Soc Lond B Biol Sci 1998; 353:821-830.

Potten, C. S., S. E. Al Barwari, W. J. Hume, and J. Searle. Circadian rhythms of presumptive stem cells in three different epithelia of the mouse. Cell Tissue Kinet. 10: 557-568, 1977.

Pourreyron C, Dumortier J, Ratineau C, Nejjari M, Beatrix O, Jacquier M F, Remy L, Chayvialle J A, Scoazec J Y. Age-dependent variations of human and rat colon myofibroblasts in culture: Influence on their functional interactions with colon cancer cells. Int J Cancer 2003; 104:28-35.

Prockop D J. Further proof of the plasticity of adult stem cells and their role in tissue repair. J Cell Biol 2003; 160:807-809.

Quaroni A, Beaulieu J F. Cell dynamics and differentiation of conditionally immortalized human intestinal epithelial cells. Gastroenterology 1997; 113:1198-1213.

Quaroni A, Hochman J. Development of intestinal cell culture models for drug transport and metabolism studies. Adv Drug Del Rev 1996; 22:3-52.

Quaroni, A., J. Q. Tian, P. Seth, and R. C. Ap. p 27(Kip1) is an inducer of intestinal epithelial cell differentiation. Am. J Physiol Cell Physiol 279: C1045-C1057, 2000.

Ramalho-Santos M, Melton D A, McMahon A P. Hedgehog signals regulate multiple aspects of gastrointestinal development. Development 2000; 127:2763-2772.

Ramalho-Santos M, Yoon S, Matsuzaki Y, Mulligan R C, Melton D A. “Stemness”: transcriptional profiling of embryonic and adult stem cells. Science 2002; 298:597-600.

Ramsanahie A, Duxbury M S, Grikscheit T C, Perez A, Rhoads D B, Gardner-Thorpe J, Ogilvie J, Ashley S W, Vacanti J P, Whang E E. Effect of GLP-2 on mucosal morphology and SGLT1 expression in tissue-engineered neointestine. Am J Physiol Gastrointest Liver Physiol 2003; 285:G1345-G1352.

Roth K A, Hertz J M, Gordon J I. Mapping enteroendocrine cell populations in transgenic mice reveals an unexpected degree of complexity in cellular differentiation within the gastrointestinal tract. J Cell Biol 1990; 1 10:1791-1801.

Rubin D C, Roth K A, Birkenmeier E H, Gordon J I. Epithelial cell differentiation in normal and transgenic mouse intestinal isografts. J Cell Biol 1991; 113:1183-1192.

Rudnicki M A. Marrow to muscle, fission versus fusion. Nat Med 2003; 9:1461-1462.

Sakakibara S, Imai T, Hamaguchi K, Okabe M, Aruga J, Nakajima K, Yasutomi D, Nagata T, Kurihara Y, Uesugi S, Miyata T, Ogawa M, Mikoshiba K, Okano H. Mouse-Musashi-1, a neural RNA-binding protein highly enriched in the mammalian CNS stem cell. Dev Biol 1996; 176:230-242.

Sandberg J W, Lau C, Jacomino M, Finegold M, Henning S J. Improving access to intestinal stem cells as a step toward intestinal gene transfer. Hum Gene Ther 1994; 5:323-329.

Sanderson I R, Ezzell R M, Kedinger M, Erlanger M, Xu Z-X, Pringault E, Leon-Robine S, Louvard D, Walker W A. Human fetal enterocytes in vitro: modulation of the phenotype by extracellular matrix. Proc Natl Acad Sci USA 1996; 93:7717-7722.

Sato M, Ahnen D J. Regional variability of colonocyte growth and differentiation in the rat. Anat Rec 1992; 233:409-414.

Schmolka I R. Artificial skin. I. Preparation and properties of pluronic F-127 gels for treatment of burns. J Biomed Mater Res 1972; 6:571-582.

Sferra T J, McNeely D, Johnson P R. Gene transfer to the intestinal tract: a new approach using selective injection of the superior mesenteric artery. Hum Gene Ther 1997; 8:681-687.

Shi, Q., S. Rafii, M. H. Wu, E. S. Wijelath, C. Yu, A. Ishida, Y. Fujita, S. Kothari, R. Mohle, L. R. Sauvage, M. A. Moore, R. F. Storb, and W. P. Hammond. Evidence for circulating bone marrow-derived endothelial cells. Blood 92: 362-367, 1998.

Simon-Assmann P, Kedinger M, De Arcangelis A, Rousseau V, Simo P. Extracellular matrix components in intestinal development. Experientia 1995; 51:883-900.

Slorach E M, Campbell F C, Dorin J R. A mouse model of intestinal stem cell function and regeneration. J Cell Sci 1999; 112 Pt 18:3029-3038.

Smith L G, Weissman I L, Heimfeld S. Clonal analysis of hematopoietic stem-cell differentiation in vivo. Proc Natl Acad Sci USA 1991 ; 88:2788-2792.

Spangrude G J, Smith L, Uchida N, Ikuta K, Heimfeld S, Friedman J, Weissman I L. Mouse hematopoietic stem cells. Blood 1991; 78:1395-1402.

Stallmach A, Hahn U, Merker H J, Hahn E G, Riecken E O. Differentiation of rat intestinal epithelial cells is induced by organotypic mesenchymal cells in vitro. Gut 1989; 30:959-970.

Stappenbeck T S, Gordon J I. Rac1 mutations produce aberrant epithelial differentiation in the developing and adult mouse small intestine. Development 2000; 127:2629-2642.

Stappenbeck T S, Mills J C, Gordon J I. Molecular features of adult mouse small intestinal epithelial progenitors. Proc Natl Acad Sci USA 2003; 100:1004-1009.

Sun Z, Wang X, Wallen R, Deng X, Du X, Hallberg E, Andersson R. The influence of apoptosis on intestinal barrier integrity in rats. Scand J Gastroenterol 1998; 33:415-422.

Szabo S, Khomenko T, Gombos Z, Deng X M, Jadus M R, Yoshida M. Review article: transcription factors and growth factors in ulcer healing. Aliment Pharmacol Ther 2000; 14 Suppl 1:33-43.

Tait I S, Evans G S, Flint N, Campbell F C. Colonic mucosal replacement by syngeneic small intestinal stem cell transplantation. Am J Surg 1994; 167:67-72.

Tavakkolizadeh A, Berger U V, Stephen A E, Kim B S, Mooney D, Hediger M A, Ashley S W, Vacanti J P, Whang E E. Tissue-engineered neomucosa: morphology, enterocyte dynamics, and SGLT1 expression topography. Transplantation 2003; 75:181-185.

Tessner T G, Cohn S M, Schloemann S, Stenson W F. Prostaglandins prevent decreased epithelial cell proliferation associated with dextran sodium sulfate injury in mice. Gastroenterology 1998; 115:874-882.

Theise N D, Nimmakayalu M, Gardner R, Illei P B, Morgan G, Teperman L, Henegariu O, Krause D S. Liver from bone marrow in humans. Hepatology 2000; 32:11-16.

Theise, N. D., S. Badve, R. Saxena, O. Henegariu, S. Sell, J. M. Crawford, and D. S. Krause. Derivation of hepatocytes from bone marrow cells in mice after radiation-induced myeloablation. Hepatology 31: 235-240, 2000.

Thomas R P, Hellmich M R, Townsend C M, Jr., Evers B M. Role of gastrointestinal hormones in the proliferation of normal and neoplastic tissues. Endocr Rev 2003; 24:571-599.

Tian, J. Q. and A. Quaroni. Involvement of p21(WAF1/Cip1) and p27(Kip1) in intestinal epithelial cell differentiation. Am. J Physiol 276: C1245-C1258, 1999.

Traber, P. G. Transcriptional regulation in intestinal development. Implications for colorectal cancer. Adv. Exp. Med. Biol. 470: 1-14, 1999.

Troyer K L, Luetteke N C, Saxon M L, Qiu T H, Xian C J, Lee D C. Growth retardation, duodenal lesions, and aberrant ileum architecture in triple null mice lacking EGF, amphiregulin, and TGF-alpha. Gastroenterology 2001; 121:68-78.

Tumbar T, Guasch G, Greco V, Blanpain C, Lowry W E, Rendl M, Fuchs E. Defining the epithelial stem cell niche in skin. Science 2004; 303:359-363.

United Network of Organ Sharing. 2001.

Van Driessche N, Shaw C, Katoh M, Morio T, Sucgang R, Ibarra M, Kuwayama H, Saito T, Urushihara H, Maeda M, Takeuchi I, Ochiai H, Eaton W, Tollett J, Halter J, Kuspa A, Tanaka Y, Shaulsky G. A transcriptional profile of multicellular development in Dictyostelium discoideum. Development 2002; 129:1543-1552.

Vanagunas A. Radiation-Induced Gastrointestinal Disease. Clin Perspectives in Gastroenterology 2001; 69-75.

Verburg M, Renes I B, Einerhand A W, Buller H A, Dekker J. Isolation-stress increases small intestinal sensitivity to chemotherapy in rats. Gastroenterology 2003; 124:660-671.

Verburg M, Renes I B, Meijer H P, Taminiau J A, Buller H A, Einerhand A W, Dekker J. Selective sparing of goblet cells and paneth cells in the intestine of methotrexate-treated rats. Am J Physiol Gastrointest Liver Physiol 2000; 279:G1037-G1047.

Vidrich A, Buzan J M, Cohn S M. Intestinal stem cells and mucosal gut development. Current Opinion in Gastroenterology 2003; 19:583-590.

Vlahos A, Yu P, Lucas C E, Ledgerwood A M. Effect of a composite membrane of chitosan and poloxamer gel on postoperative adhesive interactions. Am Surg 2001; 67:15-21.

Vogel G. Stem cells. ‘Stemness’ genes still elusive. Science 2003; 302:371.

Weiser M M, Sykes D E, Killen P D. Rat intestinal basement membrane synthesis: Epithelial versus nonepithelial contributions. Lab Invest 1990; 62:325-330.

Weiser, M. M. Intestinal epithelial cell surface membrane glycoprotein synthesis. I. An indicator of cellular differentiation. J Biol. Chem. 248: 2536-2541, 1973.

Weiss S L, Lee E A, Diamond J. Evolutionary matches of enzyme and transporter capacities to dietary substrate loads in the intestinal brush border. Proc Natl Acad Sci USA 1998; 95:2117-2121.

Welm B E, Tepera S B, Venezia T, Graubert T A, Rosen J M, Goodell M A. Sca-1(pos) cells in the mouse mammary gland represent an enriched progenitor cell population. Dev Biol 2002; 245:42-56.

Whitehead R H, Demmler K, Rockman S P, Watson N K. Clonogenic growth of epithelial cells from normal colonic mucosa from both mice and humans. Gastroenterology 1999; 117:858-865.

Whitehead R H, VanEeden P E, Noble M D, Ataliotis P, Jat P S. Establishment of conditionally immortalized epithelial cell lines from both colon and small intestine of adult H-2K^{b}-tsA58 transgenic mice. Proc Natl Acad Sci USA 1993; 90:587-591.

Wice B M, Gordon J I. Forced expression of Id-1 in the adult mouse small intestinal epithelium is associated with development of adenomas. J Biol Chem 1998; 273:25310-25319.

Wielenga V J, Smits R, Korinek V, Smit L, Kielman M, Fodde R, Clevers H, Pals S T. Expression of CD44 in Apc and Tcf mutant mice implies regulation by the WNT pathway. Am J Pathol. 1999 February; 154(2):515-23.

Wilkins H R, Ohneda K, Keku T O, D'Ercole A J, Fuller C R, Williams K L, Lund P K. Reduction of spontaneous and irradiation-induced apoptosis in small intestine of IGF-I transgenic mice. Am J Physiol Gastrointest Liver Physiol 2002; 283:G457-G464.

Williams K L, Fuller C R, Dieleman L A, DaCosta C M, Haldeman K M, Sartor R B, Lund P K. Enhanced survival and mucosal repair after dextran sodium sulfate-induced colitis in transgenic mice that overexpress growth hormone. Gastroenterology 2001; 120:925-937.

Wingender E, Chen X, Fricke E, Geffers R, Hehl R, Liebich I, Krull M, Matys V, Michael H, Ohnhauser R, Pruss M, Schacherer F, Thiele S, Urbach S. The TRANSFAC system on gene expression regulation. Nucleic Acids Res 2001; 29:281-283.

Winton D J, Ponder B A J. Stem-cell organization in mouse small intestine. Proc Roy Soc Lond B 1990; 241:13-18.

Winton D J. Stem cells in the epithelium of the small intestine and colon. Stem Cell Biology, Cold Spring Harbor Monograph Series 40, Edited by Daniel R Marshak, Ch 22, pp 2001; A515-A536.

Withers, H. R. and M. M. Elkind. Microcolony survival assay for cells of mouse intestinal mucosa exposed to radiation. Int. J Radiat. Biol. Relat Stud. Phys. Chem. Med 17: 261-267, 1970.

Wong M H, Hermiston M L, Syder A J, Gordon J I. Forced expression of the tumor suppressor adenomatosis polyposis coli protein induces disordered cell migration in the intestinal epithelium. Proc Natl Acad Sci USA 1996; 93:9588-9593.

Wright D E, Wagers A J, Gulati A P, Johnson F L, Weissman I L. Physiological migration of hematopoietic stem and progenitor cells. Science 2001; 294:1933-1936.

Wright N A, Carter J, Irwin M. The measurement of villus cell population size in the mouse small intestine in normal and abnormal states: a comparison of absolute measurements with morphometric estimators in sectioned immersion-fixed material. Cell Tissue Kinet 1989; 22:425-450.

Wright N A. Epithelial stem cell repertoire in the gut: clues to the origin of cell lineages, proliferative units and cancer. Int J Exp Pathol 2000; 81 :117-143.

Wulf G G, Luo K L, Jackson K A, Brenner M K, Goodell M A. Cells of the hepatic side population contribute to liver regeneration and can be replenished with bone marrow stem cells. Haematologica 2003; 88:368-378.

Wulf, G. G., K. Luo, K. A. Jackson, M. K. Brenner, and M. A. Goodell. Cells of the hepatic side population (SP) contribute to liver regeneration and are replenishable from bone marrow stem cells. Am. J. Physiol. 2001.

Yang H, Finaly R, Teitelbaum D H. Alteration in epithelial permeability and ion transport in a mouse model of total parenteral nutrition. Crit Care Med 2003; 31:1118-1125.

Yang H, Wildhaber B E, Teitelbaum D H. 2003 Harry M. Vars Research Award. Keratinocyte growth factor improves epithelial function after massive small bowel resection. JPEN J Parenter Enteral Nutr 2003; 27:198-206.

Yang Q, Bermingham N A, Finegold M, Zoghbi H Y. Requirement of Math1 for secretory cell lineage commitment in the mouse intestine. Science 2001; 294:2155-2158.

Yang, Q., N. A. Bermingham, M. Finegold, and H. Y. Zoghbi. Math1 governs secretory versus absorptive cell lineage commitment in the mouse intestine. Am. J. Physiol. In press: 2001.

Yatabe Y, Tavare S, Shibata D. Investigating stem cells in human colon by using methylation patterns. Proc Natl Acad Sci USA 2001; 98:10839-10844.

Ye H, Kelly T F, Samadani U, Lim L, Rubio S, Overdier D G, Roebuck K A, Costa R H. Hepatocyte nuclear factor 3/fork head homolog 11 is expressed in proliferating epithelial and mesenchymal cells of embryonic and adult tissues. Mol Cell Biol 1997; 17:1626-1641.

Yeh E T, Zhang S, Wu H D, Korbling M, Willerson J T, Estrov Z. Transdifferentiation of human peripheral blood CD34+-enriched cell population into cardiomyocytes, endothelial cells, and smooth muscle cells in vivo. Circulation 2003; 108:2070-2073.

Yeh K-Y, Holt P R. Ontogenic timing mechanism initiates the expression of rat intestinal sucrase activity. Gastroenterology 1986; 90:520-526.

Yuen T, Wurmbach E, Pfeffer R L, Ebersole B J, Sealfon S C. Accuracy and calibration of commercial oligonucleotide and custom cDNA microarrays. Nucleic Acids Res 2002; 30:e48.

Zambrowicz B P, Imamoto A, Fiering S, Herzenberg L A, Kerr W G, Soriano P. Disruption of overlapping transcripts in the ROSA βgeo 26 gene trap strain leads to widespread expression of β-galactosidase in mouse embryos and hematopoietic cells. Proc Natl Acad Sci USA 1997; 94:3789-3794.

Zhou, S., J. D. Schuetz, K. D. Bunting, A. M. Colapietro, J. Sampath, J. J. Morris, I. Lagutina, G. C. Grosveld, M. Osawa, H. Nakauchi, and B. P. Sorrentino. The ABC transporter Bcrp1/ABCG2 is expressed in a wide variety of stem cells and is a molecular determinant of the side-population phenotype. Nat. Med. 7: 1028-1034, 2001.

Zimmermann, J. W. and R. M. Schultz. Analysis of gene expression in the preimplantation mouse embryo: Use of mRNA differential display. Proc. Natl. Acad. Sci. USA 91: 5456-5460, 1994.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An isolated mammalian gastrointestinal stem cell, said cell characterized as comprising the following:
CD45 negative;
Collagen IV negative; and
Msi-1 positive.

2. The cell of claim 1, comprising one or more characteristics selected from the group consisting of Hes-1 positive, CD34 negative, Thy1.2 negative, Sca1 negative, c-kit negative, EphB2 positive, CD44 positive, Math-1 positive, Hath-1 positive, and Brcp1 positive.

3. The cell of claim 1, wherein said cell is isolated from the gastrointestinal tract.

4. The cell of claim 1, wherein said cell is isolated from the stomach, large intestine, or small intestine.

5. The cell of claim 1, wherein said cell is isolated from the stomach, duodenum, jejunum, ileum, colon, cecum, or rectum.

6. The cell of claim 1, wherein said cell is isolated from the stomach.

7. The cell of claim 1, wherein said cell is isolated from the duodenum.

8. The cell of claim 1, wherein the cell is isolated from the jejunum.

9. The cell of claim 1, wherein said cell is isolated from the ileum.

10. The cell of claim 1, wherein said cell is isolated from the colon.

11. The cell of claim 1, wherein said cell is isolated from the cecum.

12. The cell of claim 1, wherein said cell is isolated from the rectum.

13. The cell of claim 1, wherein said cell is isolated from an adult mammal.

14. The cell of claim 13, wherein the adult mammal is a human or mouse.

15. The cell of claim 1, wherein said cell is isolated from an infant mammal.

16. The cell of claim 15, wherein the infant mammal is a human or mouse.

17. The cell of claim 1, wherein said cell is further defined as being in a suspension of cells that are substantially single in nature.

18. The cell of claim 17, wherein the suspension of substantially single cells is further defined as being substantially free of organoids.

19. The cell of claim 17, wherein the suspension of substantially single cells is further defined as being substantially free of mesenchymal tissue, mesenchymal cells, or both.

20. The cell of claim 17, wherein the suspension of substantially single cells is suitable for a cell sorting method.

21. The cell of claim 17, wherein the suspension of substantially single cells is obtained from the method of:
providing gastrointestinal tissue from a mammal;
subjecting the tissue to mechanical disruption, chemical disruption, enzymatic digestion, or a combination of two or more thereof, wherein said subjecting step generates the suspension of substantially single cells.

22. The cell of claim 21, wherein the mechanical disruption comprises cutting, chopping, slicing, shaking, or shearing the gastrointestinal tissue.

23. The cell of claim 21, wherein the chemical disruption comprises disruption with EDT A, mannitol, or a mixture thereof.

24. The cell of claim 21, wherein the enzymatic digestion comprises dispase, collagenase, trypsin, pancreatin, or a mixture thereof.

25. The method of claim 21, further comprising the step of removing mucus from the gastrointestinal tissue.

26. An isolated mammalian gastrointestinal stem cell that is CD45 negative; Collagen IV negative; and Msi-1 positive, said cell obtained from the method of:
providing gastrointestinal tissue from a mammal; and
subjecting the tissue to mechanical disruption, chemical disruption, enzymatic digestion, or a combination of two or more thereof, wherein said subjecting step provides the isolated stem cell.

27. A method of isolating a mammalian gastrointestinal stem cell, comprising the steps of:
providing gastrointestinal tissue from the mammal; and
subjecting the intestinal tissue to at least one disruption method, wherein the subjecting step provides a plurality of single cells, said plurality of single cells defined as substantially free of cell aggregates, wherein the single cells are defined as being:
CD45 negative;
Collagen IV negative; and
Msi-1 positive.

28. The method of claim 27, further comprising the step of removing mucus from the gastrointestinal tissue.

29. The method of claim 27, wherein the providing step comprises removing tissue from the jejunum of the mammal.

30. A method of treating intestinal tissue in an individual in need thereof, comprising:
obtaining isolated stem cells comprising the following characteristics:
CD45 negative;
Collagen IV negative; and
Msi-1 positive; and
delivering a therapeutically effective amount of the stem cells to the intestine in need of treatment.

31. The method of claim 30, wherein the intestine of the individual is in need of therapy due to necrotizing enterocolitis, infarction, injury, insulin-dependent diabetes, cancer, hormone deficiency; clotting disorder, phenylketonuria, a metabolic disorder, inflammatory bowel disease (IBD), or ulcer.

32. The method of claim 30, wherein the hormone deficiency comprises deficiency of growth hormone.

33. The method of claim 30, wherein the metabolic disorder is a urea cycle disorder.

34. The method of claim 30, wherein the individual was treated and/or is being treated with therapy that is damaging to the intestine.

35. The method of claim 34, wherein the therapy damaging to the intestine comprises cancer therapy.

36. The method of claim 34, wherein the therapy damaging to the intestine comprises therapy with at least one nonsteroidal anti-inflammatory drug.

37. The method of claim 35, wherein the cancer therapy is chemotherapy, radiation, surgery, or a combination of two or more thereof.

38. The method of claim 30, wherein the isolated stem cells are delivered to the intestine in need thereof prior to the therapy that is damaging to the intestine.

39. The method of claim 30, wherein the isolated stem cells are delivered to the intestine in need thereof concomitant with the therapy that is damaging to the intestine.

40. The method of claim 30, wherein the isolated stem cells are delivered to the intestine in need thereof subsequent to the therapy that is damaging to the intestine.

41. The method of claim 30, wherein the obtaining step comprises:
providing gastrointestinal tissue from a mammal;
subjecting the tissue to mechanical disruption, chemical disruption, enzymatic digestion, or a combination of two or more thereof, wherein said subjecting step generates a suspension of isolated stem cells.

42. The method of claim 41, wherein the mammal is the same species as the individual.

43. The method of claim 30, wherein the stem cells are isolated from the individual in need thereof.

44. The method of claim 30, wherein the stem cells are isolated from an individual which is not the individual in need thereof.

45. A method of treating intestinal tissue in an individual in need thereof, comprising:
obtaining at least one isolated stem cell comprising the following characteristics:
CD45 negative;
Collagen IV negative; and
Msi-1 positive; and
introducing to the stem cell a polynucleotide encoding a gene product that is therapeutic for the intestinal tissue; and
delivering a therapeutically effective amount of the stem cell comprising the polynucleotide to the intestine in need of treatment.

46. The method of claim 45, wherein the stem cells are isolated from the individual in need thereof.

47. The method of claim 45, wherein the stem cells are isolated from an individual which is not the individual in need thereof.

48. A method for preventing, treating, or both, intestinal damage in an individual with cancer, comprising:
administering radiation, chemotherapy, or both, to the individual;
obtaining at least one isolated mammalian gastrointestinal stem cell comprising the following characteristics:
CD45-negative;
collagen IV-negative; and
Msi-1-positive; and
delivering a therapeutically effective amount of the at least one isolated stem cell to the intestine of the individual.

49. The method of claim 48, wherein the stem cell is delivered to the individual prior to administration of the radiation and/or chemotherapy step.

50. The method of claim 48, wherein the stem cell is delivered to the individual concomitant with administration of the radiation and/or chemotherapy step.

51. The method of claim 48, wherein the stem cell is delivered to the individual subsequent to administration of the radiation and/or chemotherapy step.

52. The method of claim 48, wherein the stem cell is obtained from the individual prior to the administering of the radiation and/or chemotherapy step.

53. The method of claim 48, wherein the stem cell is obtained from another individual or individuals that are not the individual with cancer.
